(19) United States
(12) Patent Application Publication
(10) Pub. No.: US 2015/0024947 A1
(43) Pub. Date: Jan. 22, 2015

(54) SYSTEMS AND METHODS FOR DISTINGUISHING BETWEEN AUTISM SPECTRUM DISORDERS (ASD) AND NON-ASD DEVELOPMENTAL DELAY

(71) Applicant: SynapDx Corporation, Lexington, MA (US)

(72) Inventors: Stanley Letovsky, Milton, MA (US); Theresa Tribble, Lexington, MA (US); Stanley N. Lapidus, Bedford, NH (US)

(21) Appl. No.: 14/472,344

(22) Filed: Aug. 28, 2014

Related U.S. Application Data
(63) Continuation of application No. 13/841,470, filed on Mar. 15, 2013.
(60) Provisional application No. 61/682,633, filed on Aug. 13, 2012.

(51) Int. Cl. C12Q 1/68

(52) U.S. Cl. CPC C12Q 1/6883; C12Q 2600/158; C12Q 2600/112
USPC 506/2; 506/9

(57) ABSTRACT

Methods and systems are presented herein to distinguish children with Autism Spectrum Disorders (ASD) from those with other forms of developmental delay (DD) based on patterns of gene expression levels in blood.

### RELATED APPLICATIONS

This application claims the benefit of U.S. patent application Ser. No. 13/841,470 filed on Mar. 15, 2013, which claims the benefit of U.S. Provisional Application No. 61/682,633 filed on Aug. 13, 2012; the entirety of each of which is herein incorporated by reference.

### FIELD OF THE INVENTION

This invention relates generally to systems and methods for identifying Autism Spectrum Disorders (ASD) in an individual.

### BACKGROUND

Autism Spectrum Disorders (ASD) are pervasive developmental disorders which are being diagnosed at increasing rates, likely due to some combination of increased awareness by clinicians and a true rise in incidence. These disorders are characterized by reciprocal social interaction deficits, language difficulties, and repetitive behaviors and restrictive interests that manifest during the first 3 years of life. While there are currently no effective medical therapies that target the core symptoms of ASD, behavioral therapy is effective at reducing the severity of symptoms, and at better integrating a child diagnosed with an ASD into the family, the school and the community. Increasingly, data point to the value of commencing behavioral therapy at an early age; accordingly, the AAP has emphasized the importance of early diagnosis of ASD. Since 2007 American Academy of Pediatrics (AAP) guidelines have recommended regular screening for developmental delays and ASD specifically; yet recent data show that although the average age at which parents begin to suspect an ASD in their child is 20 months, the average age of diagnosis is 48 months.

The etiology of ASD is poorly understood but is thought to be multifactorial, with both genetic and environmental factors contributing to disease development. A variety of types of genetic mutations have been associated with ASD, including copy number variations, rare single-nucleotide variations and common single nucleotide polymorphisms. To date only a few causative genetic loci have been reliably identified, and these individually account for less than 1% of ASD cases, and collectively account for less than 20%.

From a clinical perspective, an important challenge is assessing whether children require specialist referral for an autism diagnosis and treatment plan rather than, or in addition to, referral to an early intervention program when a developmental delay is suspected. Delayed referral may explain the CDC's recent observation that only 18% of children who end up with an ASD diagnosis are identified by age 36 months. An objective test with good sensitivity would improve the ability to identify these children earlier, when therapeutic intervention is more effective.

### SUMMARY

Methods and systems are presented herein to distinguish children with Autism Spectrum Disorders (ASD) from those with other forms of developmental delay (DD) based on patterns of gene expression levels in blood. It is found that blood gene expression biomarkers are useful in providing an objective method of identifying children at increased risk for an ASD within populations with symptoms of developmental delay.

In one aspect, the invention is directed to a method for distinguishing between or among at least two conditions for diagnosis and/or risk assessment of an individual suspected of having or observed as having atypical development, wherein the at least two conditions comprise autism spectrum disorder (ASD) and developmental delay not due to autism spectrum disorder (DD), the method comprising the steps of: measuring an expression level of each of one or more genes of a sample obtained from the individual; identifying, by a processor of a computing device, at least one of: (i) the existence (or non-existence) of ASD in the individual as opposed to at least one other condition indicative of atypical development and exclusive of ASD, wherein the at least one other condition comprises DD, said identifying based at least in part on the measured expression level of the one or more genes (e.g., distinguishing between ASD and DD in the individual based at least in part on the measured expression level of the one or more genes); and (ii) a likelihood the individual has (or does not have) ASD as opposed to at least one other condition indicative of atypical development and exclusive of ASD, wherein the at least one other condition comprises DD, said identifying based at least in part on the measured expression level of the one or more genes.

In some embodiments, the individual is independently suspected of having (e.g., by a medical practitioner) or is independently observed to have (e.g., by a medical practitioner) atypical development, said independent suspicion or observation having been made prior to the identifying step. In some embodiments, the method comprises identifying, by the processor of the computing device, the existence of ASD in the individual as opposed to DD. In some embodiments, the method comprises identifying, by the processor of the computing device, a risk score quantifying the likelihood the individual has ASD as opposed to at least one other condition, wherein the at least one other condition comprises DD. In some embodiments, the method comprises identifying, by the processor of the computing device, a risk score quantifying the likelihood the individual has ASD as opposed to DD.

In some embodiments, measuring the expression level of the one or more genes comprises assembling, by a processor of a computing device, multiple, fragmented sequence reads. In some embodiments, measuring the expression level of the one or more genes comprises conducting an assay using a high-throughput sequencer apparatus (e.g., using a technology that parallelizes the sequencing process, e.g., using RNA-Seq technology, e.g., using a “next generation” sequencer). In some embodiments, conducting the assay comprises performing at least one technique selected from the group consisting of single-molecule real-time sequencing (e.g., Pacific Bio), ion semiconductor sequencing (e.g., Ion Torrent sequencing), pyrosequencing (e.g., 454), sequencing by synthesis (e.g., Illumina), sequencing by ligation (e.g., SOLiD sequencing), and chain termination sequencing (e.g., microfluidic Sanger sequencing).

In some embodiments, measuring the expression level of the one or more genes comprises obtaining RNA from the sample, creating cDNA from the RNA, and identifying the cDNA by hybrid capture. In some embodiments, measuring the expression level of the one or more genes comprises sequencing expressed RNA from the sample. In some embodiments, measuring the expression level of the one or more genes comprises determining a copy number of expressed RNA in the sample. In some embodiments, the RNA is mRNA.

In some embodiments, the one or more genes comprise (or consist of) at least one gene whose expression level is higher or lower (e.g., by a statistically significant amount) in a subject with ASD relative to its expression level in a subject who does not have ASD. In some embodiments, the one or more genes comprise (or consist of) at least one gene whose expression level is higher or lower (e.g., to a statistically significant degree) in a subject with ASD relative to its expression level in a subject with DD.

In some embodiments, the sample is a blood sample. In some embodiments, the sample comprises white blood cells. In some embodiments, the sample comprises plasma or cerebrospinal fluid.

In some embodiments, the individual has been identified by a medical practitioner as displaying atypical behavior prior to the identifying step. In some embodiments, the individual is five years old or less (e.g., three years old or less, 24 months old or less, or 20 months old or less).

In some embodiments, the method further comprises the step of: performing a chromosomal microarray (CMA) test (e.g., an array comparative genomic hybridization, aCGH, test) with a sample obtained from the individual, wherein the identifying step comprises: identifying, by the processor of the computing device, at least one of: (i) the existence of ASD in the individual as opposed to at least one other condition, wherein the at least one other condition comprises DD, based at least in part on (a) the measured expression level of the one or more genes and (b) the CMA test; and (ii) a relative likelihood the individual has ASD as opposed to at least one other condition, wherein the at least one other condition comprises DD, based at least in part on (a) the measured expression level of the one or more genes and (b) the CMA test. In some embodiments, the CMA test determines the presence or absence of a potentially causative genetic lesion associated with ASD.

In some embodiments, the at least one other condition comprises one or more members selected from the group consisting of Autism (AU), No ASD, General Population with Typical Development (TD), and Atypical (e.g., as defined in the CHARGE study, Childhood Autism Risk from Genetics and the Environment). In some embodiments, developmental delay not due to autism spectrum disorder (DD) means non-Autism (AU) and non-ASD with (i) score of 69 or lower on Mullen, score of 69 or lower on Vineland, and score of 14 or lower on SCQ, or (ii) score of 69 or lower on either Mullen or Vineland and within half a standard deviation of cutoff value on the other assessment (score 77 or lower).

In some embodiments, measuring the expression level of the one or more genes comprises measuring the expression level of each of one or more members (e.g., at least one, at least three, at least five, at least eight, at least ten, at least fifteen, or at least 20 members) selected from the group consisting of C20orf173, TRPM5, TPM2, CCNE2, CKAP2L, CAND2, MTRNR2L3, LDLRAP1, ASPM, ZDHHC15, RASL10B, ST8SIA1, CLEC12B, MARCKSL1, SHCBP1, DEPDC1, TSHR, NCAPG, RPLP2, CENPA, SORBS3, MCM10, HELLS, RNF208, E2F8, PTK7, GRM3, CPSF1, and CDHR1.

In some embodiments, the identifying step comprises computing a score using a gene expression signature, wherein the measured expression level of the one or more genes (e.g., normalized, un-normalized, ratioed, un-ratioed) is/are used as input in the gene expression signature. In some embodiments, the score is a numerical risk score and the gene expression signature differentiates between two categories (e.g., ASD and DD) or differentiates among three or more categories. In some embodiments, the gene expression signature is an optimal differentiating hyperplane. In some embodiments, the gene expression signature differentiates between two categories (e.g., ASD and DD), and the AUC (area under a curve of a graph displaying normalized true positive and false positive rates of differential diagnosis based at least on the measured expression level of the one or more genes and a binary indicator (e.g., ASD vs. DD)) is 60% or greater. In some embodiments, AUC is 63% or greater (e.g., 65% or greater). In some embodiments, the method has a sensitivity of at least about 90% and a specificity of at least about 20% (e.g., at least about 23%, or at least about 24%). In some embodiments, the gene expression signature is determined based upon a plurality of gene expression profiles for individuals with ASD and a plurality of gene expression profiles for individuals with DD. In some embodiments, the gene expression signature is determined by applying differential expression analysis to downsample RNA sequencing data. In some embodiments, the gene expression signature is determined by performing propensity score sampling to obtain subsample sets balanced for age and gender.

In another aspect, the invention is directed to a system for distinguishing between or among at least two conditions for diagnosis and/or risk assessment of an individual suspected of having or observed as having atypical development, wherein the at least two conditions comprise autism spectrum disorder (ASD) and developmental delay not due to autism spectrum disorder (DD), the system comprising: a diagnostics kit comprising testing instruments for measuring an expression level of each of one or more genes of a sample obtained from the individual; and a non-transitory computer-readable medium having instructions stored thereon, wherein the instructions, when executed by a processor, cause the processor to: identify at least one of: (i) the existence (or non-existence) of ASD in the individual as opposed to at least one other condition indicative of atypical development and exclusive of ASD, wherein the at least one other condition comprises DD, said identifying based at least in part on the measured expression level of the one or more genes (e.g., distinguish between ASD and DD in the individual based at least in part on the measured expression level of the one or more genes); and (ii) a likelihood the individual has (or does not have) ASD as opposed to at least one other condition indicative of atypical development and exclusive of ASD, wherein the at least one other condition comprises DD, said identifying based at least in part on the measured expression level of the one or more genes.

In some embodiments, the diagnostics kit is an in vitro diagnostics kit. In some embodiments, the diagnostics kit is an RNA-Seq diagnostics kit. In some embodiments, the individual is independently suspected of having (e.g., by a medical practitioner) or is independently observed to have (e.g., by a medical practitioner) atypical development.

In some embodiments, the instructions cause the processor to identify the existence of ASD in the individual as opposed to DD (e.g., distinguish between ASD and DD). In some embodiments, the instructions cause the processor to identify a risk score quantifying the likelihood the individual has ASD as opposed to at least one other condition, wherein the at least one other condition comprises DD. In some embodiments, the instructions cause the processor to identify a risk score quantifying the likelihood the individual has ASD as opposed to DD.

In some embodiments, the measured expression level of the one or more genes comprises processed output of a high-throughput sequencer apparatus (e.g., processed using a technology that parallelizes the sequencing process, e.g., using RNA-Seq technology, e.g., using a “next generation” sequencer). In some embodiments, the high-throughput sequencer apparatus is configured to perform at least one technique selected from the group consisting of single-molecule real-time sequencing (e.g., Pacific Bio), ion semiconductor sequencing (e.g., Ion Torrent sequencing), pyrosequencing (e.g., 454), sequencing by synthesis (e.g., Illumina), sequencing by ligation (e.g., SOLiD sequencing), and chain termination sequencing (e.g., microfluidic Sanger sequencing). In some embodiments, the one or more genes comprise (or consist of) at least one gene whose expression level is higher or lower (e.g., by a statistically significant amount) in a subject with ASD relative to its expression level in a subject who does not have ASD. In some embodiments, the one or more genes comprise (or consist of) at least one gene whose expression level is higher or lower (e.g., to a statistically significant degree) in a subject with ASD relative to its expression level in a subject with DD.

In some embodiments, the sample is a blood sample. In some embodiments, the sample comprises white blood cells. In some embodiments, the sample comprises plasma or cerebrospinal fluid.

In some embodiments, the individual is five years old or less (e.g., three years old or less, 24 months old or less, or 20 months old or less).

In some embodiments, the system further comprises a kit for performing a chromosomal microarray (CMA) test (e.g., an array comparative genomic hybridization, aCGH, test) with a sample obtained from the individual, wherein the instructions cause the processor to identify at least one of: (i) the existence of ASD in the individual as opposed to at least one other condition, wherein the at least one other condition comprises DD, based at least in part on (a) the measured expression level of the one or more genes and (b) the CMA test; and (ii) a relative likelihood the individual has ASD as opposed to at least one other condition, wherein the at least one other condition comprises DD, based at least in part on (a) the measured expression level of the one or more genes and (b) the CMA test. In some embodiments, the CMA test determines the presence or absence of a potentially causative genetic lesion associated with ASD.

In some embodiments, the at least one other condition comprises one or more members selected from the group consisting of Autism (AU), No ASD, General Population with Typical Development (TD), and Atypical (e.g., as defined in the CHARGE study, Childhood Autism Risk from Genetics and the Environment). In some embodiments, developmental delay not due to autism spectrum disorder (DD) means non-Autism (AU) and non-ASD with (i) score of 69 or lower on Mullen, score of 69 or lower on Vineland, and score of 14 or lower on SCQ, or (ii) score of 69 or lower on either Mullen or Vineland and within half a standard deviation of cutoff value on the other assessment (score 77 or lower).

In some embodiments, the one or more genes comprises one or more members (e.g., at least one, at least three, at least five, at least eight, at least ten, at least fifteen, or at least 20 members) selected from the group consisting of C20orf173, TRPM5, TPM2, CCNE2, CKAP2L, CAND2, MTRNR2L3, LDLRAP1, ASPM, ZDHHC15, RASL10B, ST8SIA1, CLEC12B, MARCKSL1, SHCBP1, DEPDC1, TSHR, NCAPG, RPLP2, CENPA, SORBS3, MCM10, HELLS, RNF208, E2F8, PTK7, GRM3, CPSF1, and CDHR1.

In some embodiments, the instructions cause the processor to identify a score using a gene expression signature, wherein the measured expression level of the one or more genes (e.g., normalized, un-normalized, ratioed, un-ratioed) is/are used as input in the gene expression signature. In some embodiments, the score is a numerical risk score and the gene expression signature differentiates between two categories (e.g., ASD and DD) or differentiates among three or more categories. In some embodiments, the gene expression signature is an optimal differentiating hyperplane. In some embodiments, the gene expression signature differentiates between two categories (e.g., ASD and DD), and the AUC (area under a curve of a graph displaying normalized true positive and false positive rates of differential diagnosis based at least on the measured expression level of the one or more genes and a binary indicator (e.g., ASD vs. DD)) is 60% or greater. In some embodiments, the AUC is 63% or greater (e.g., 65% or greater). In some embodiments, the system has a sensitivity of at least about 90% and a specificity of at least about 20% (e.g., at least about 23%, or at least about 24%). In some embodiments, the gene expression signature is based upon a plurality of gene expression profiles for individuals with ASD and a plurality of gene expression profiles for individuals with DD.

In some embodiments, the gene expression signature reflects application of differential expression analysis to downsample RNA sequencing data. In some embodiments, the gene expression signature reflects performance of propensity score sampling to obtain subsample sets balanced for age and gender.

In another aspect, the invention is directed to a non-transitory computer-readable medium having instructions stored thereon, wherein the instructions, when executed by a processor, cause the processor to: access measurements of an expression level of each of one or more genes of a sample obtained from an individual suspected of having or observed as having atypical development; and identify at least one of: (i) the existence (or non-existence) of ASD in the individual as opposed to at least one other condition indicative of atypical development and exclusive of ASD, wherein the at least one other condition comprises DD, said identifying based at least in part on the measured expression level of the one or more genes (e.g., distinguish between ASD and DD in the individual based at least in part on the measured expression level of the one or more genes); and (ii) a likelihood the individual has (or does not have) ASD as opposed to at least one other condition indicative of atypical development and exclusive of ASD, wherein the at least one other condition comprises DD, said identifying based at least in part on the measured expression level of the one or more genes.

In another aspect, the invention is directed to a method of treating an individual suspected of having or observed as having atypical development, the method comprising the steps of: obtaining a sample from the individual; measuring an expression level of each of one or more genes of the sample; identifying, by a processor of a computing device, at least one of: (i) the existence of ASD in the individual as opposed to at least one other condition indicative of atypical development and exclusive of ASD, wherein the at least one other condition comprises DD, said identifying based at least in part on the measured expression level of the one or more genes (e.g., distinguishing between ASD and DD in the individual based at least in part on the measured expression level of the one or more genes); and (ii) a likelihood the individual has ASD as opposed to at least one other condition indicative of atypical development and exclusive of ASD, wherein the at least one other condition comprises DD, said identifying based at least in part on the measured expression level of the one or more genes; and administering therapy to the individual for ASD. In some embodiments, the therapy is behavioral therapy. In some embodiments, the therapy comprises administration of a therapeutic substance.

In some embodiments, the individual is independently suspected of having (e.g., by a medical practitioner) or is independently observed to have (e.g., by a medical practitioner) atypical development, said independent suspicion or observation having been made prior to the identifying step.

In some embodiments, the method comprises identifying, by the processor of the computing device, the existence of ASD in the individual as opposed to DD. In some embodiments, the method comprises identifying, by the processor of the computing device, a risk score quantifying the likelihood the individual has ASD as opposed to at least one other condition, wherein the at least one other condition comprises DD. In some embodiments, the method comprises identifying, by the processor of the computing device, a risk score quantifying the likelihood the individual has ASD as opposed to DD.

In some embodiments, measuring the expression level of the one or more genes comprises assembling, by a processor of a computing device, multiple, fragmented sequence reads. In some embodiments, measuring the expression level of the one or more genes comprises conducting an assay using a high-throughput sequencer apparatus (e.g., using a technology that parallelizes the sequencing process, e.g., using RNA-Seq technology, e.g., using a “next generation” sequencer). In some embodiments, conducting the assay comprises performing at least one technique selected from the group consisting of single-molecule real-time sequencing (e.g., Pacific Bio), ion semiconductor sequencing (e.g., Ion Torrent sequencing), pyrosequencing (e.g., 454), sequencing by synthesis (e.g., Illumina), sequencing by ligation (e.g., SOLiD sequencing), and chain termination sequencing (e.g., microfluidic Sanger sequencing).

In some embodiments, measuring the expression level of the one or more genes comprises obtaining RNA from the sample, creating cDNA from the RNA, and identifying the cDNA by hybrid capture. In some embodiments, measuring the expression level of the one or more genes comprises sequencing expressed RNA from the sample. In some embodiments, measuring the expression level of the one or more genes comprises determining a copy number of expressed RNA in the sample. In some embodiments, the RNA is mRNA.

In some embodiments, the one or more genes comprise (or consist of) at least one gene whose expression level is higher or lower (e.g., by a statistically significant amount) in a subject with ASD relative to its expression level in a subject who does not have ASD. In some embodiments, the one or more genes comprise (or consist of) at least one gene whose expression level is higher or lower (e.g., to a statistically significant degree) in a subject with ASD relative to its expression level in a subject with DD.

In some embodiments, the sample is a blood sample. In some embodiments, the sample comprises white blood cells. In some embodiments, the sample comprises plasma or cerebrospinal fluid.

In some embodiments, the individual has been identified by a medical practitioner as displaying atypical behavior prior to the identifying step. In some embodiments, the individual is five years old or less (e.g., three years old or less, 24 months old or less, or 20 months old or less).

In some embodiments, the method further comprises the step of: performing a chromosomal microarray (CMA) test (e.g., an array comparative genomic hybridization, aCGH, test) with a sample obtained from the individual, wherein the identifying step comprises: identifying, by the processor of the computing device, at least one of: (i) the existence of ASD in the individual as opposed to at least one other condition, wherein the at least one other condition comprises DD, based at least in part on (a) the measured expression level of the one or more genes and (b) the CMA test; and (ii) a relative likelihood the individual has ASD as opposed to at least one other condition, wherein the at least one other condition comprises DD, based at least in part on (a) the measured expression level of the one or more genes and (b) the CMA test. In some embodiments, the CMA test determines the presence or absence of a potentially causative genetic lesion associated with ASD.

In some embodiments, the at least one other condition comprises one or more members selected from the group consisting of Autism (AU), No ASD, General Population with Typical Development (TD), and Atypical (e.g., as defined in the CHARGE study, Childhood Autism Risk from Genetics and the Environment). In some embodiments, developmental delay not due to autism spectrum disorder (DD) means non-Autism (AU) and non-ASD with (i) score of 69 or lower on Mullen, score of 69 or lower on Vineland, and score of 14 or lower on SCQ, or (ii) score of 69 or lower on either Mullen or Vineland and within half a standard deviation of cutoff value on the other assessment (score 77 or lower).

In some embodiments, measuring the expression level of the one or more genes comprises measuring the expression level of each of one or more members (e.g., at least one, at least three, at least five, at least eight, at least ten, at least fifteen, or at least 20 members) selected from the group consisting of C20orf173, TRPM5, TPM2, CCNE2, CKAP2L, CAND2, MTRNR2L3, LDLRAP1, ASPM, ZDHHC15, RASL10B, ST8SIA1, CLEC12B, MARCKSL1, SHCBP1, DEPDC1, TSHR, NCAPG, RPLP2, CENPA, SORBS3, MCM10, HELLS, RNF208, E2F8, PTK7, GRM3, CPSF1, and CDHR1.

In some embodiments, the identifying step comprises computing a score using a gene expression signature, wherein the measured expression level of the one or more genes (e.g., normalized, un-normalized, ratioed, un-ratioed) is/are used as input in the gene expression signature. In some embodiments, the score is a numerical risk score and the gene expression signature differentiates between two categories (e.g., ASD and DD) or differentiates among three or more categories. In some embodiments, the gene expression signature is an optimal differentiating hyperplane. In some embodiments, the gene expression signature differentiates between two categories (e.g., ASD and DD), and the AUC (area under a curve of a graph displaying normalized true positive and false positive rates of differential diagnosis based at least on the measured expression level of the one or more genes and a binary indicator (e.g., ASD vs. DD)) is 60% or greater. In some embodiments, the AUC is 63% or greater (e.g., 65% or greater). In some embodiments, the method has a sensitivity of at least about 90% and a specificity of at least about 20% (e.g., at least about 23%, or at least about 24%).

In some embodiments, the gene expression signature is determined based upon a plurality of gene expression profiles for individuals with ASD and a plurality of gene expression profiles for individuals with DD. In some embodiments, the gene expression signature is determined by applying differential expression analysis to downsample RNA sequencing data. In some embodiments, the gene expression signature is determined by performing propensity score sampling to obtain subsample sets balanced for age and gender.

In some embodiments (of any of the methods or systems herein), the identifying accounts for one or more demographic parameters and/or biophysical measurements of the individual.

The description of elements of the embodiments with respect to one aspect of the invention can be applied to another aspect of the invention as well. For example, features described in a claim depending from an independent method claim may be applied, in another embodiment, to an independent system claim.

### BRIEF DESCRIPTION OF THE FIGURES

The foregoing and other objects, aspects, features, and advantages of the present disclosure will become more apparent and better understood by referring to the following description taken in conjunction with the accompanying drawings, in which:

FIG. 1 is a flow chart of a method of determining a score, likelihood, or diagnosis of ASD, rather than non-ASD DD, in accordance with an illustrative embodiment.

FIG. 2 is a schematic flow chart showing a method of classifier signature training and/or use, in accordance with an illustrative embodiment.

FIGS. 3A, 3B, and 3C are flow charts of a method of classifier signature training and/or use, in accordance with an illustrative embodiment.

FIGS. 4A and 4B are flow charts of a method of classifier signature training and/or use, in accordance with an illustrative embodiment.

FIG. 5 is an exemplary cloud computing environment 500 for use with the systems and methods described herein, in accordance with an illustrative embodiment.

FIG. 6 is an example of a computing device 600 and a mobile computing device 650 that can be used to implement the techniques described in this disclosure.

FIG. 7 is a graph depicting a gene expression signature of biological processes enriched in differentially expressed genes between Autism Spectrum Disorder (ASD) and Development Delay (DD).

The features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements.

### DETAILED DESCRIPTION

Methods and systems are presented herein to distinguish children with Autism Spectrum Disorders (ASD) from those with other forms of developmental delay (DD) based on patterns of gene expression levels in blood.

Ribonucleic acid (RNA) includes, but is not limited to, messenger RNA (mRNA) which determines the specific amino acid sequence in the protein that is produced and non-coding RNA (ncRNA) which does not produce a mature protein. Although ncRNA don't encode functional protein, ncRNAs are never-the-less important for many biological functions. Non-limiting examples of ncRNAs include long noncoding RNA (e.g. Xist) which can modulate gene expression, ribosomal RNA (rRNA) which is the central component of the ribosome's protein-manufacturing machinery, transfer RNA (tRNA) which mediates recognition of the codon and provides the corresponding amino acid, small nuclear RNA (snRNA) which is involved in the processing of pre-mRNA in the nucleus, and microRNA (miRNA) and small interfering RNA (siRNA) which modulate gene expression through complementary mRNA binding (i.e. the process of RNA interference or RNAi) and/or target methylation.

In the study example presented herein below, mRNA samples isolated from blood from children ages 2-5 years diagnosed with ASD (n=174) or DD (n=96) were sequenced using next-generation sequencing of RNA (RNASeq) to measure blood gene expression levels. The samples were divided into a training set and a holdout set. Genes that differed between ASD and DD in the training set were selected by t-test and used to develop a support vector machine (SVM) signature. The performance of the signature was assessed on the holdout set.

The classifiers showed an ability to partially distinguish the two groups based on gene expression. The mean AUC of the ROC curve for the holdout set was 65.5±3.8%. Selecting a threshold of 90% sensitivity for the signature risk score resulted in a specificity of 23.9±8.0% (95% confidence interval: [12.6, 39.0]). Gene categories that significantly differed between ASD and DD samples included cell cycle and immune processes.

This study example includes determination of a classification signature for ASD versus DD using peripheral blood samples. These results provide evidence that blood gene expression biomarkers are useful in providing an objective method of identifying children at increased risk for an ASD within populations with symptoms of developmental delay.

Autism Spectrum Disorders (ASD) are pervasive developmental disorders which are being diagnosed at increasing rates, due to some combination of increased awareness by clinicians and a true rise in incidence. These disorders are characterized by reciprocal social interaction deficits, language difficulties, and repetitive behaviors and restrictive interests that manifest during the first 3 years of life. While there are currently no effective medical therapies that target the core symptoms of ASD, behavioral therapy is effective at reducing the severity of symptoms, and at better integrating a child diagnosed with an ASD into the family, the school and the community. Increasingly, data point to the value of commencing behavioral therapy at an early age; accordingly, the AAP has emphasized the importance of early diagnosis of ASD. Since 2007 American Academy of Pediatrics (AAP) guidelines have recommended regular screening for developmental delays and ASD specifically; yet recent data show that although the average age at which parents begin to suspect an ASD in their child is 20 months, the average age of diagnosis is 48 months.

The etiology of ASD is poorly understood but is thought to be multifactorial, with both genetic and environmental factors contributing to disease development. A variety of types of genetic mutations have been associated with ASD, including copy number variations, rare single-nucleotide variations and common single nucleotide polymorphisms. To date only a few causative genetic loci have been reliably identified, and these individually account for less than 1% of ASD cases, and collectively account for less than 20%.

An advantage of assessing mRNA expression is that the cellular levels of an mRNA are influenced not only by its DNA sequence but also by environmental and physiological factors that can influence RNA transcription, processing and stability.

Identification of gene expression patterns characteristic of ASD can provide biomarkers to aid in early detection and treatment of ASD. Prior studies involve distinguishing ASD from typically developing (TD) controls. However, prior studies have not addressed whether gene expression patterns can distinguish ASD subjects from those with other types of developmental delay (DD) likely to be considered as alternative diagnoses in initial clinical evaluations of children suspected of development problems.

### Study Example

### Study Samples

This study used blood samples from subjects enrolled in the ongoing CHARGE (Childhood Autism Risks from Genetics and the Environment) study, collected between October 2005 and March 2011. CHARGE is being performed in accordance with the latest version of the Declaration of Helsinki, and ICH Guidelines. The study was approved by the appropriate ethics committee. One or both parents, or a legal guardian provided written informed consent.

CHARGE enrolls children with ASD, children with developmental delay but not ASD, and also typically developing controls. All subjects were between 24 and 61 months of age; gender was 24% female overall (see Table 1). Self-reported race and ethnicity were diverse and well-balanced across diagnostic groups.

Participants in the CHARGE study were assigned to one of 8 diagnostic categories based on cutoffs on their scores on the ADOS, ADI-R, Mullens, Vineland, and SCQ tests. (See Supplemental Table 1 for detailed definitions of the diagnostic categories). Since the goal of this current work was to compare expression patterns from ASD subjects to non-ASD subjects with developmental concerns, i.e., those most likely to be considered as candidates for an ASD diagnosis during an initial evaluation, we aggregated the CHARGE diagnostic groups into a set of ASD cases, comprising the CHARGE categories autism (CH-AU) and autism spectrum disorder (CH-ASD), and a set of DD controls, comprising the CHARGE categories delayed development (excluding Down Syndrome) (CH-DD), atypical (CH-Atypical), and enrolled as delayed but tested typical (CH-DD2TD) (see Table 1).

CHARGE categories excluded from this study were: the No ASD group, the typical development group, Down Syndrome subjects, and incompletely evaluated subjects. The No ASD group had been diagnosed as being on the autism spectrum by community practitioners but failed to meet study criteria for ASD. Because of this inconsistency in diagnosis, this group was not useful either for training a signature or assessing its performance, and so was excluded. Down Syndrome subjects were excluded because they would normally be identified at a much earlier age than the age of ASD diagnosis; also Down Syndrome is easy to diagnose by gene expression, so inclusion of these subjects would have tended to inflate signature performance. In addition, 30 samples from included categories were lost to process failures during RNASeq, or failed quality control (QC) criteria. Supplemental Materials Table 1 shows category definitions and sample numbers before and after exclusion and QC; QC criteria are in Supplemental Methods.

Samples were randomized into 19 sequencing batches to preserve global gender and diagnosis frequencies within each batch. Ten sequencing batches were used to form a training set, called CHARGE 1 (n=153), while the remaining 9 batches were used to form a holdout set (CHARGE 2) (n=117) (see Table 1).

The ASD and DD groups constructed from the CHARGE sample were not perfectly balanced with respect to age and gender. For example, the ASD group was 21.3% female, while the DD group was 26% female (Table 1). By chance this imbalance was enhanced to 21% and 28.3% in the CHARGE 1 subset. Age was reasonably well balanced overall (mean 3.8 vs. 3.7 years in ASD and DD), but slightly less balanced, and in opposite directions, in the CHARGE 1 and 2 subsets.

### Gene Expression Measurement and Data Analysis

Gene expression was measured using RNA Sequencing (RNASeq), a process in which RNA molecules are sequenced on a next-generation sequencing instrument and the number of fragments mapping to each gene is counted to create a histogram of relative gene abundance.

A machine learning training and evaluation pipeline was developed to train support vector machine (SVM) gene expression signatures. To prevent the signatures from being misled by gene expression signals caused by age or gender differences in the composition of the ASD and DD groups, we used propensity score sampling to repeatedly subsample from the full training and holdout sets subsamples balanced for age and gender, and for equal numbers of cases and controls. We trained a signature on each of 30 balanced subsamples of the training set, and assessed each signature's performance on 30 balanced subsamples of the holdout set. From each trial, we computed signature performance metrics, including area under the receiver operator characteristic curve (AUC) and specificity at the 90% sensitivity point. These metrics were averaged over all the subsamples. Importantly, no information from the holdout set was ever used to train the signatures; in particular, the selection of genes used as predictive features was based solely on the training set subsample used in any given trial.

We used the gene ontology biological process (GO-BP) gene sets (available on the World Wide Web at geneontology.org) and the Ingenuity Pathway Analyzer (Ingenuity® Systems, available on the World Wide Web at ingenuity.com) to suggest possible mechanistic relationships for the differentially expressed genes. A more detailed description of the laboratory and computational methods is included below.

### Results

The signatures used in this study produce a numeric risk score when applied to a given subject. In order to classify a subject as higher or lower risk for ASD a threshold score value must be chosen as the dividing line between lower and higher risk, and this choice can be more or less conservative, depending on one's preference for sensitivity over specificity, or equivalently, for false positive over false negative errors. The area under the ROC curve is a measure of signature performance across all possible thresholds that varies between 0 and 100%, with 50% representing a random classifier, and 100% representing a perfect classifier. The mean AUC for signatures trained on age and gender balanced subsamples of CHARGE 1 and tested on balanced subsamples of CHARGE 2 was 65.5±3.8%, which is significantly different from chance performance at a P<0.001 level. Choosing a classification threshold that favors high (90%) sensitivity for detecting ASD yielded a mean specificity of 23.9%.±8.0%, which was significantly different from chance performance at a P<0.05 level. Using CHARGE 2 samples for training and testing on CHARGE 1 gave a mean AUC of 65.4%±3.8% (P<0.001) and a mean specificity of 24.3±7.6% (P<0.05).

The positive predictive value (PPV) was 68.5% and negative predictive value (NPV) was 58% for classifiers trained on CHARGE 1 and tested on CHARGE 2. In contrast to AUC, sensitivity and specificity, PPV and NPV depend on the prevalence of ASD within the CHARGE study (64.4%), which was influenced by the recruiting strategy and may not reflect clinical prevalence in an intended-use population.

Identification of Genes and Gene Categories that Differ Between ASD and DD

Table 2 shows the 30 genes with the most significant difference in gene expression between ASD and DD in the full dataset in this study; a more complete list is in the Supplemental Materials Table S2. This list should not be interpreted as a list of “autism genes.” No causal role in the etiology of the disease for these genes has been demonstrated here, only correlation with the ASD/DD distinction. Moreover, changes in gene expression patterns often affect many genes, not all of them related to a specific biological process. Sampling and technical variation can also affect whether a gene makes it into a top-30 or top-300 list.

A strategy for assigning biological meaning to gene lists resulting from differential expression studies is to ask whether sets of genes involved in a particular biological process are behaving similarly, presumably due to co-regulation at the level of pathways or cellular programs. We used the Gene Ontology, a curated catalog that groups genes into functional categories, to identify biological process categories that showed statistically significant enrichment in differentially expressed genes. Numerous categories were significant at a false discovery rate threshold of 30%, meaning that 70% of these categories are expected to be “true discoveries.” The significant categories are summarized in Table 3, where they are grouped thematically. Key themes that are apparent include cell cycle, immune processes and neurological development. We also used the Ingenuity Pathway Analysis (IPA) tool from Ingenuity (Redwood City, Calif.) to identify canonical pathways associated with the differentially expressed genes. This provides an independent approach to biological interpretation using a different underlying database of gene function data, as well as different statistical methods. The IPA results highlighted pathways related to cancer (i.e., cell cycle) as well as immune and axonal guidance pathways.

### Discussion

In this study, we identified a gene expression signature derived from blood that can classify from a mixed population of ASD and DD subjects those at higher risk for ASD. The mean ROC AUC was 65%, with a specificity of 24% at the 90% sensitivity threshold. Biological processes that showed enrichment in differentially expressed genes between ASD and DD included cell cycle, neuronal and immune-related responses.

It is perhaps surprising that a disorder of the brain is detectable in blood. Without wishing to be bound by any particular theory, it is possible that alterations in gene expression in the brain (perhaps due to genetic variations) may either directly or indirectly affect gene expression in other tissues, including blood. The effect could also relate to perturbations of specific functions of blood. There may be a possible immune or autoimmune component of ASD, and immune gene categories have been identified herein as differentially expressed in ASD.

The present study differs from prior autism gene expression studies in several important respects. While some studies have looked at brain tissue, transformed blood cell lines, or purified white cells, the CHARGE blood samples used here were acquired by routine phlebotomy using PAXgene tubes, which have been cleared for clinical use by the FDA, thus providing a straightforward path to sample collection in clinical settings.

Some previous ASD gene expression studies have focused on narrowly defined ASD subpopulations with particular genetic lesions; although such populations may have more distinctive expression signatures and may provide insights into disease mechanism, they are less clinically relevant due to the rarity of those particular mutations.

All previous ASD gene expression studies have used microarrays to measure gene expression, whereas this study used next-generation sequencing (RNASeq). The RNASeq process produces millions of short DNA sequence reads that can be counted to quantify the levels of mRNA in a sample. The simplicity of this counting process avoids the complex normalizations required for microarray data, and may make RNASeq less susceptible to the batch effects and technical artifacts that plague microarray data.

It is interesting to compare the quantitative performance of the example gene expression signature described for this study to that of more traditional genetic testing. Genetic diagnostic testing for children with ASD began initially with G-banded karyotype testing in the late 1970s. Today, chromosomal microarrays (CMA), also called array comparative genomic hybridization (aCGH), is recommended for diagnosis of individuals with unexplained ASD or DD/ID to uncover the cause of the condition. CMA arrays identify potentially causative genetic lesions in 15-20% of children with ASD or DD/ID. The specificity of aCGH for distinguishing ASD from DD does not appear to have been reported in the literature, but would be expected to be only moderate, since many risk alleles have variable expressivity and may lead to either ASD or DD. CMA thus has lower sensitivity and unknown specificity, while our expression signature, with a suitable choice of threshold, has higher sensitivity and lower specificity. In certain embodiments, performance is improved by combining both types of information.

From a clinical perspective, an important challenge is assessing whether children require specialist referral for an autism diagnosis and treatment plan rather than, or in addition to, referral to an early intervention program when a developmental delay is suspected. Delayed referral may explain the CDC's recent observation that only 18% of children who end up with an ASD diagnosis are identified by age 36 months. An objective test with high sensitivity increases ability to identify these children earlier, when therapeutic intervention is more effective.

### Tables

TABLE 1
Patient demographics and disease characteristics

| | CHARGE 1 | | | CHARGE 2 | | | CHARGE 1 + 2 | | |
|---|---|---|---|---|---|---|---|---|---|
| | ASD | DD | All | ASD | DD | All | ASD | DD | All |
| ASD | 32 | — | — | 24 | — | — | 56 | — | — |
| AU | 68 | — | — | 50 | — | — | 118 | — | — |
| All | 100 | — | — | 74 | — | — | 174 | — | — |
| Atypical | — | 8 | — | — | 5 | — | — | 13 | — |
| DD | — | 31 | — | — | 22 | — | — | 53 | — |
| DD to TD | — | 14 | — | — | 16 | — | — | 30 | — |
| All | — | 53 | — | — | 43 | — | — | 96 | — |
| Total | — | — | 153 | — | — | 117 | — | — | 270 |
| Female | 21 (21.0) | 15 (28.3) | 36 (23.5) | 16 (21.6) | 10 (23.3) | 26 (22.2) | 37 (21.3) | 25 (26.0) | 62 (23.0) |
| n (%) | | | | | | | | | |
| Male, n (%) | 79 (79.0) | 38 (71.7) | 117 (76.5) | 58 (78.4) | 33 (76.7) | 91 (77.8) | 137 (78.7) | 71 (74.0) | 208 (77.0) |
| Mean age, yrs | 3.7 (0.7) | 3.9 (0.7) | 3.8 (0.7) | 3.8 (0.8) | 3.6 (0.8) | 3.7 (0.8) | 3.8 (0.8) | 3.7 (0.8) | 3.8 (0.8) |
| (±SD) | | | | | | | | | |
| Mean Mullens | 63.7 (19.2) | 67.8 (16.5) | 65.1 (18.4) | 63.1 (19.0) | 71.1 (19.2) | 66.0 (19.4) | 63.4 (19.1) | 69.3 (17.70 | 65.5 (18.8) |
| score | | | | | | | | | |
| (±SD)^{b} | | | | | | | | | |
| Mean Vineland | 66.2 (13.60) | 70.5 (13.7 | 67.7 (13.7) | 60.7 (9.8) | 71.0 (13.0) | 64.5 (12.1) | 63.9 (12.4) | 70.7 (13.4) | 66.3 (13.1) |
| score (±SD)^{c} | | | | | | | | | |

^{a}Column labels are diagnostic classifications used in the analysis and first rows are diagnostic classifications from CHARGE, described in detail in Supplemental Materials Table 1
^{b}Mullens Early Learning Composite Score
^{c}Vineland Composite Score
ASD = autism spectrum disorder;.
AU = strict autism;
DD = delayed development;
DD to TD = referred as DD but tested as typical

TABLE 2
Top 30 Genes by ASD/DD differential expression in entire dataset

| Gene Symbol | Descriptions | −log₁₀ p(T)^{a} | log₂ FC^{b} |
|---|---|---|---|
| C20orf173 | Chromosome 20 open reading frame 173 | 4.8 | −0.43 |
| TRPM5 | Transient receptor potential cation channel, subfamily M, member 5 | 4.4 | 0.45 |
| TPM2 | Tropomyosin 2 (beta) | 4.4 | 0.29 |
| CCNE2 | Cyclin E2 | 3.9 | −0.25 |
| CKAP2L | Cytoskeleton associated protein 2-like | 3.8 | −0.41 |
| CAND2 | Cullin-associated and neddylation-dissociated 2 (putative) | 3.8 | 0.28 |
| MTRNR2L3 | MT-RNR2-like 3 | 3.7 | −0.33 |
| LDLRAP1 | Low density lipoprotein receptor adaptor protein 1 | 3.7 | 0.16 |
| ASPM | Asp (abnormal spindle) homolog, microcephaly associated (Drosophila) | 3.7 | −0.40 |
| ZDHHC15 | Zinc finger, DHHC-type containing 15 | 3.7 | 0.38 |
| RASL10B | RAS-like, family 10, member B | 3.6 | 0.35 |
| ST8SIA1 | ST8 alpha-N-acetyl-neuraminide alpha-2,8-sialyltransferase 1 | 3.6 | −0.22 |
| CLEC12B | C-type lectin domain family 12, member B | 3.6 | −0.43 |
| MARCKSL1 | MARCKS-like 1 | 3.6 | 0.14 |
| SHCBP1 | SHC SH2-domain binding protein 1 | 3.5 | −0.34 |
| DEPDC1 | DEP domain containing 1 | 3.5 | −0.43 |
| TSHR | Thyroid stimulating hormone receptor | 3.4 | −0.45 |
| NCAPG | Non-SMC condensin I complex, subunit G | 3.4 | −0.34 |
| RPLP2 | Ribosomal protein, large, P2 | 3.4 | 0.17 |
| CENPA | Centromere protein A | 3.4 | −0.40 |
| SORBS3 | Sorbin and SH3 domain containing 3 | 3.4 | 0.14 |
| MCM10 | Minichromosome maintenance complex component 10 | 3.4 | −0.42 |
| HELLS | Helicase, lymphoid-specific | 3.3 | −0.23 |
| RNF208 | Ring finger protein 208 | 3.3 | 0.27 |
| E2F8 | E2F transcription factor 8 | 3.3 | −0.40 |
| PTK7 | PTK7 protein tyrosine kinase 7 | 3.3 | 0.25 |
| GRM3 | Glutamate receptor, metabotropic 3 | 3.3 | −0.34 |
| CPSF1 | Cleavage and polyadenylation specific factor 1, 160 kDa | 3.3 | 0.15 |
| CDHR1 | Cadherin-related family member 1 | 3.2 | 0.27 |

^{a}−log₁₀ p(T) is the negative base 10 logarithm of the P-value of the T-statistic, which is moderated to augment the variance with a component that depends on mean expression levels, thereby depressing the significance of low expressors which tend to have higher variance.
^{b}log₂ FC is the average fold-change between the ASD and DD groups in log2 expression units; positive values mean higher in the ASD group.

TABLE 1
Significantly differentially expressed Gene Ontology categories (FDR < 0.3), grouped into thematic supercategories.
Categories are ordered by decreasing significance; supercategories by their most significant category.

| Supercategory | Categories |
|---|---|
| Cell cycle | Cell cycle phase, regulation of mitotic cell cycle, regulation of mitosis, regulation of nuclear division, |
| | negative regulation of cell cycle process, mitotic cell cycle spindle checkpoint, regulation of chromosome |
| | segregation, establishment of mitotic spindle localization, chromosome segregation, G2/M transition |
| | checkpoint & 40 others |
| Cytoskeleton | Cell-cell junction assembly, regulation of cell-cell adhesion, regulation of microtubule-based process, |
| | microtubule cytoskeleton organization, negative regulation of actin filament depolymerization, |
| | microtubule polymerization or depolymerization, positive regulation of microtubule polymerization or |
| | depolymerization |
| Development | Endothelial cell migration, regulation of smooth muscle cell apoptosis, negative regulation of epithelial |
| | cell differentiation, negative regulation of fibroblast proliferation, regulation of myoblast differentiation, |
| | oocyte maturation, embryonic pattern specification, myoblast differentiation, negative regulation of cell |
| | development, negative regulation of muscle organ development & 3 others |
| Immune | Regulation of cytokine secretion, positive regulation of interferon-gamma biosynthetic process, positive |
| | regulation of interleukin-12 biosynthetic process, negative regulation of leukocyte activation, positive |
| | regulation of cytokine secretion, response to protozoan, defense response to protozoan, response to |
| | defenses of other organism involved in symbiotic interaction, response to host, response to host defenses |
| | & 14 others |
| Metabolic | Tetrahydrofolate metabolic process, prostaglandin biosynthetic process, prostanoid biosynthetic process, |
| | ribonucleoside diphosphate metabolic process, internal protein amino acid acetylation, regulation of |
| | cholesterol metabolic process, regulation of hydrogen peroxide metabolic process, regulation of |
| | cholesterol biosynthetic process, carbohydrate phosphorylation, glycerol-3-phosphate metabolic process |
| | & 18 others |
| Other | Regulation of transcription from RNA polymerase I promoter, temperature homeostasis, multicellular |
| | organismal homeostasis, response to gravity, cotranslational protein targeting to membrane, negative |
| | regulation of protein complex assembly, cellular response to inorganic substance, cellular response to |
| | metal ion, negative regulation of heart contraction, regulation of protein binding & 1 others |
| Protein catabolism | Response to endoplasmic reticulum stress, cellular response to unfolded protein, endoplasmic reticulum |
| | unfolded protein response, negative regulation of proteasomal ubiquitin-dependent protein catabolic |
| | process, proteolysis involved in cellular protein catabolic process, protein K6-linked ubiquitination, ER to |
| | Golgi vesicle-mediated transport |
| Transport | Sequestering of metal ion, inorganic anion transport, anion transport, organic anion transport, negative |
| | regulation of nucleocytoplasmic transport, quaternary ammonium group transport, regulation of |
| | mitochondrial membrane permeability, gas transport |
| DNA damage | Postreplication repair, G2/M transition DNA damage checkpoint, double-strand break repair via |
| | homologous recombination, recombinational repair, response to X-ray, positive regulation of DNA repair, |
| | response to ionizing radiation, response to radiation, DNA damage response, signal transduction resulting |
| | in induction of apoptosis, DNA damage response, signal transduction by p53 class mediator & 4 others |
| Neural | Negative regulation of gliogenesis, dopamine metabolic process, regulation of glial cell differentiation, |
| | regulation of gliogenesis, neurotransmitter secretion, positive regulation of neuron differentiation, neuron |
| | differentiation, regulation of neurotransmitter levels |
| Blood | Response to fluid shear stress, platelet activation, regulation of vascular permeability |
| Signaling | Positive regulation of tyrosine phosphorylation of STAT protein, regulation of retinoic acid receptor |
| | signaling pathway, positive regulation of calcium-mediated signaling, I-kappaB phosphorylation, cellular |
| | response to steroid hormone stimulus, regulation of calcium-mediated signaling, SMAD protein signal |
| | transduction, induction of positive chemotaxis, negative regulation of steroid hormone receptor signaling |
| | pathway, response to amino acid stimulus & 5 others |
| Post-translational | Histone acetylation, internal peptidyl-lysine acetylation, peptidyl-lysine acetylation, peptidyl-lysine |
| modification | modification, protein amino acid acylation, protein amino acid acetylation, protein modification by small |
| | protein conjugation |
| Apoptosis | Regulation of muscle cell apoptosis, induction of apoptosis, induction of programmed cell death |

### Supplemental Information

### Detailed Methods

### RNA Isolation

Total RNA from 2.5 mL of blood acquired from CHARGE participants using the Qiagen PAXgene™ Blood RNA System (Qiagen, Hilden, Germany) was frozen at −80° C. for up to 2.4 years (mean time between draw and isolation was 7±8 months) and subsequently isolated using QiaGen's PAXgene Blood RNA Kit, per manufacturer's instructions, in approximate order of collection date. For initial quality control, we required total RNA samples to have an RNA integrity number (RIN)≧7.5 and an RNA concentration of ≧17 ng/μL. 1 ul of a 1:100 dilution of ERCC RNA Spike-In Control Mix 1 or 2 (Ambion/Life Technologies, Carlsbad, Calif., USA) was added to each sample (850 ng) as an internal standard.

### Library Preparation and Sequencing

For sequencing, subjects' RNA samples were randomized into 19 batches that preserved global gender and diagnosis frequencies within each batch. Sequencing libraries were prepared using TruSeq RNA Sample Prep Kit v2 (Illumina Inc., San Diego, Calif., USA) per manufacturer's instructions. The TruSeq kit includes a polyA selection step that enriches for mRNA. 850 ng of total RNA was used from each patient's sample. Only libraries with fragment sizes of ≧250 and ≦350 and >80% inserts were accepted for sequencing. Cluster generation and sequencing were performed using the TruSeq SR Cluster Kit v3 (Illumina) per manufacturer's instructions. Sequence barcodes were attached to the samples to allow multiplexing of samples within sequencer lanes. Barcoded libraries from 24 samples were mixed and the mixture was loaded onto each of the 8 lanes of one flowcell of a HiSeq 2000 (Illumina), yielding a net coverage of ⅓ of a lane per sample. Fifty-one base single-ended sequencing was performed, followed by 7 bases of barcode sequence. Average raw yield was 175 million reads per lane.

### RNA-Seq Data Analysis

Base calling and barcode demultiplexing were performed using Illumina's CASAVA v1.8.2 on an Amazon Cloud linux instance. Barcodes were demultiplexed with zero allowed errors per barcode, which equates to an expected 0.02% rate of assigning reads to the wrong sample, based on the intrinsic base error rate of Illumina sequencing. Reads were analyzed using the Tuxedo RNAseq pipeline64, which includes the Bowtie aligner v1.4.1 (accessed via the hypertext transfer protocol bowtie-bio.sourceforge.net/index.shtml) and the Cufflinks transcript quantitation program v1.3.0 (accessed via the hypertext transfer protocol cufflinks.cbcb.umd.edu).

Bowtie was used to align sequence reads to the human transcriptome. A reference transcriptome was used that included only a single transcript per gene based on observed quantitation anomalies in Cufflinks in the presence of multiple transcripts. The longest transcript for each gene was selected from Illumina's hg19 reference assembly gene annotation. Average aligned yield was 53.3 million reads per sample. A minimum of 30 million mapped reads per library were required to accept a sample for further analysis. Cufflinks was used to convert the reads to gene-specific fragments per kilobase per million (FPKM). FPKM were renormalized to counts per gene, which were then further normalized for differences in coverage between samples by downsampling each sample according to a scale factor estimated using the method of Anders and Huber. This yielded a total counts per sample that provided robustly similar coverage of most genes across samples. The use of downsampling, rather than scaling, preserves both mean and variance properties of the normalized counts, and also eliminates coverage effects on presence/absence of low expressors.

### Quality Control

Of the 30 samples in the diagnostic categories of interest that failed, 18 failed due to not meeting pre-specified laboratory QC cutoffs discussed in the RNA Isolation and Library preparation and Sequencing sections; these included samples in a batch that failed due to a protocol error. Five additional samples failed because they fell below the pre-specified 30 million aligned reads per sample cutoff. Four samples were excluded because they exceeded a pre-specified cutoff for RMS deviation from the study grand median per gene expression; this check was designed to exclude outlier samples that likely were affected by unknown technical issues. Three samples were excluded because the apparent gender of the sample disagreed with the subject information. Sample gender was assessed using a simple gene-expression-based gender classifier which is normally extremely reliable (AUC=100%). These samples are presumed to have been swapped at some point in the sample handling custody chain. Since a swap would only be detectable by this means only if the swapped samples were of different genders, the observed swap rate of 1.4% suggests an estimated actual swap rate affecting 4% of samples.

### Signature Training

A machine learning training and evaluation pipeline was developed in MatLab using the support vector machine (SVM) routines in the Statistics Toolbox v.7.5. In each signature training run, the best 300 predictive genes were selected by t-test and clustered into 7 clusters using k-means clustering, to reduce redundancy and enhance common signals. Propensity matching was used to create gender and age balanced training and holdout sets by fitting a logistic regression model to predict diagnostic group (ASD or DD) as a function of age and gender, and binning the predicted probabilities into 5 equal-sized bins. In each bin, all of the samples from the less frequent diagnostic group were retained, and an equal number from the more frequent group were selected at random. This process was repeated over numerous iterations of sampling, training and testing to produce average performance estimates for the classifiers.

### Gene Category Analysis

We used the gene ontology biological process (GO-BP) gene sets (available on the World Wide Web at geneontology.org) to suggest possible mechanistic relationships for the differentially expressed genes. The gene X subject expression data matrix was converted into a matrix of ranks, with 1 denoting the subject with the lowest expression value of a gene, and 270 (the number of subjects) denoting the highest. For each category with at least 10 expressed genes in the reference, and for each subject, a two-sided Kolmogorov-Smirnov (KS) test (MATLAB kstest2 function) was used to compare the distribution of ranks of genes in the category for that subject to a uniform distribution, in order to detect excess over- or under-expression of genes in the category in that subject (i.e., did that subject have unusually high or low ranks of genes in the category). The negative log of the KS probability was signed according to whether the median rank was below or above expectation. This procedure yielded a subject X category matrix of signed category over/under-expression significance. The distributions of these numbers for each category were then compared across the two diagnostic groups (ASD and DD) using KS. The process was repeated for 1,000 random permutations of the diagnostic labels to create a null distribution of KS significances for each gene, which was then used to convert the observed KS significance to a p-value for each category. These p-values were then adjusted for multiple comparisons using the false-discovery rates method of Story via MATLAB's mafdr function. Categories were thresholded at a q-value of 0.3 to identify a set of categories such that 70% of them are expected to be truly differentially expressed.

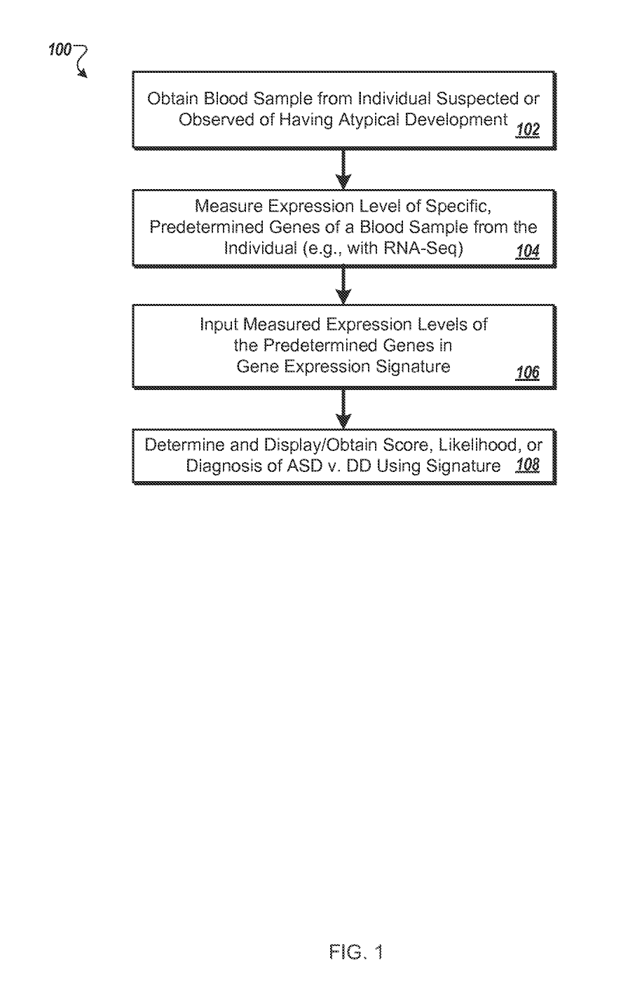

Canonical pathways analysis was used to identify pathways from Ingenuity's IPA library of canonical pathways that were most enriched with differentially expressed genes. The moderated T-statistic was used as a fold-change-like input to IPA. The significance of the association between the T-statistics from the data set and each canonical pathway was measured in 2 ways: 1) A ratio of the number of genes from the data set that map to the pathway divided by the total number of genes that map to the canonical pathway is displayed; 2) Fisher's exact test was used to calculate a p-value determining the probability that the association between the genes in the dataset and the canonical pathway is explained by chance alone. The false-discovery-rate adjusted p-values and ratios are shown in FIG. 1.

SUPPLIMENTAL TABLE 1
CHARGE diagnostic categories

| Category | N | N | |
|---|---|---|---|
| (symbol) | initial^{a} | included^{b} | |
| Autism | 129 | 118 | Autism Disorder criteria are 1) must meet autism cutoff on Communication + Social Interaction Total in |
| (CH-AU) | | | ADOS and 2) meets cutoff values on all 4 sections of ADI-R (A. Social Interaction, B. Communication, |
| | | | C. Patterns of Behavior, D. Abnormality of Development at ≦36 mo). |
| ASD | 63 | 56 | ASD criteria are 1) child does not meet criteria for autism; 2) meets ASD cutoff on Communication + |
| (CH-ASD) | | | Social Interaction Total in ADOS; and 3) (a) meets cutoff value for A. Social Interaction and B. |
| | | | Communication or (b) meets cutoff value for A. Social Interaction or B. Communication and is within 2 |
| | | | points of cutoff value on A. Social Interaction or B. Communication (whichever did not meet cutoff |
| | | | value) in ADI-R or (c) is within 1 point of cutoff value on A. Social Interaction and B. Communication; |
| | | | and 4) meets cutoff value on section D. Abnormality of Development at ≦36 mo in ADI-R. |
| No ASD | 34 | — | No ASD (applicable to AUs (children with prior diagnosis of autism or ASD from Regional Center) or |
| | | | non-AU children who complete AU protocol (for non-AUs ADOS is administered first and if meet |
| | | | criteria on ADOS then ADIR is administered)) does not meet criteria for Autism or ASD; subsets: |
| | | | “Met 1 cutoff” means that met criteria for autism or ASD on either ADOS only or ADIR only. |
| General | 93 | — | Typical development (non-AU groups only) criteria are 1) score of 70 or higher on Mullen; 2) score of |
| population | | | 70 or higher on Vineland; AND 3) score of 14 or lower on SCQ (clinician judgment may substitute SCQ |
| with typical | | | score). |
| development | | | |
| (TD) | | | |
| Atypical | 13 | 13 | Atypical development/Mild delays (non-AU groups only) criteria are 1) does not meet criteria for typical |
| | | | development and 2) does not meet criteria for delayed development. |
| Delayed | 63 | 53 | Delayed development (non-AU groups only) criteria are 1) score 69 or lower on Mullen; 2) score of 69 |
| development | | | or lower on Vineland; AND 3) score of 14 or lower on SCQ (clinician judgment may substitute SCQ |
| (CH-DD) | | | score). Also DD if has score of 69 or lower on either Mullen or Vineland and is within half a standard |
| | | | deviation of cutoff value on the other assessment (score 77 or lower). Down Syndrome subjects are |
| | | | counted elsewhere. |
| Enrolled as | 32 | 30 | |
| DD but | | | |
| tested | | | |
| typical | | | |
| Down | 19 | — | |
| Syndrome | | | |
| Incomplete | 6 | — | |
| Evaluation | | | |

^{a}N initial indicates the number of subjects having PAXgene blood samples.
^{b}N final reflects the number of subjects used in the analysis. Reduced numbers relative to the initial values are due to quality control failures

SUPPLEMENTAL TABLE 2
Differentially Expressed Genes: top 300 ASD/DD differentially
expressed genes by −log(p(T)) based on full dataset.

| Gene Symbol | Description | −log₁₀(p(T)) | log₂FC |
|---|---|---|---|
| C20orf173 | chromosome 20 open reading frame 173 | 4.8 | −0.43 |
| TRPM5 | transient receptor potential cation channel, subfamily M, | 4.4 | 0.45 |
| | member 5 | | |
| TPM2 | tropomyosin 2 (beta) | 4.4 | 0.29 |
| CCNE2 | cyclin E2 | 3.9 | −0.25 |
| CKAP2L | cytoskeleton associated protein 2-like | 3.8 | −0.41 |
| CAND2 | cullin-associated and neddylation-dissociated 2 (putative) | 3.8 | 0.28 |
| MTRNR2L3 | MT-RNR2-like 3 | 3.7 | −0.33 |
| LDLRAP1 | Low density lipoprotein receptor adaptor protein 1 | 3.7 | 0.16 |
| ASPM | Asp (abnormal spindle) homolog, microcephaly associated | 3.7 | −0.40 |
| | (Drosophila) | | |
| ZDHHC15 | Zinc finger, DHHC-type containing 15 | 3.7 | 0.38 |
| RASL10B | RAS-like, family 10, member B | 3.6 | 0.35 |
| ST8SIA1 | ST8 alpha-N-acetyl-neuraminide alpha-2,8- | 3.6 | −0.22 |
| | sialyltransferase 1 | | |
| CLEC12B | C-type lectin domain family 12, member B | 3.6 | −0.43 |
| MARCKSL1 | MARCKS-like 1 | 3.6 | 0.14 |
| SHCBP1 | SHC SH2-domain binding protein 1 | 3.5 | −0.34 |
| DEPDC1 | DEP domain containing 1 | 3.5 | −0.43 |
| TSHR | Thyroid stimulating hormone receptor | 3.4 | −0.45 |
| NCAPG | Non-SMC condensin I complex, subunit G | 3.4 | −0.34 |
| RPLP2 | Ribosomal protein, large, P2 | 3.4 | 0.17 |
| CENPA | Centromere protein A | 3.4 | −0.40 |
| SORBS3 | Sorbin and SH3 domain containing 3 | 3.4 | 0.14 |
| MCM10 | Minichromosome maintenance complex component 10 | 3.4 | −0.42 |
| HELLS | Helicase, lymphoid-specific | 3.3 | −0.23 |
| RAF208 | Ring finger protein 208 | 3.3 | 0.27 |
| E2F8 | E2F transcription factor 8 | 3.3 | −0.40 |
| PTK7 | PTK7 protein tyrosine kinase 7 | 3.3 | 0.25 |
| GRM3 | Glutamate receptor, metabotropic 3 | 3.3 | −0.34 |
| CPSF1 | Cleavage and polyadenylation specific factor 1, 160 kDa | 3.3 | 0.15 |
| CDHR1 | Cadherin-related family member 1 | 3.2 | 0.27 |
| RPS28 | Ribosomal protein S28 | 3.2 | 0.17 |
| APBB1 | Amyloid beta (A4) precursor protein-binding, family B, | 3.2 | 0.16 |
| | member 1 (Fe65) | | |
| RPL18 | Ribosomal protein L18 | 3.2 | 0.15 |
| MDS2 | Myelodysplastic syndrome 2 translocation associated | 3.2 | 0.23 |
| TRIP13 | Thyroid hormone receptor interactor 13 | 3.2 | −0.37 |
| STMN3 | Stathmin-like 3 | 3.2 | 0.16 |
| TCEAL3 | Transcription elongation factor A (SII)-like 3 | 3.2 | 0.16 |
| UBA52 | Ubiquitin A-52 residue ribosomal protein fusion product 1 | 3.2 | 0.20 |
| BUB1B | Budding uninhibited by benzimidazoles 1 homolog beta | 3.2 | −0.30 |
| | (yeast) | | |
| C5 | Complement component 5 | 3.2 | −0.18 |
| ST13 | Suppression of tumorigenicity 13 (colon carcinoma) | 3.2 | 0.09 |
| | (Hsp70 interacting protein) | | |
| KIF11 | Kinesin family member 11 | 3.1 | −0.26 |
| ABHD3 | Abhydrolase domain containing 3 | 3.1 | −0.14 |
| PLEKHB1 | Pleckstrin homology domain containing, family B | 3.1 | 0.17 |
| | (evectins) member 1 | | |
| SIGIRR | Single immunoglobulin and toll-interleukin 1 receptor | 3.1 | 0.12 |
| | (TIR) domain | | |
| ALS2CL | ALS2 C-terminal like | 3.1 | 0.20 |
| CEP55 | Centrosomal protein 55 kDa | 3.1 | −0.37 |
| SOX8 | SRY (sex determining region Y)-box 8 | 3.1 | 0.27 |
| CAPN5 | Calpain 5 | 3.0 | 0.17 |
| XIRP2 | Xin actin-binding repeat containing 2 | 3.0 | 0.35 |
| ITGA1 | Integrin, alpha 1 | 3.0 | −0.27 |
| DEPDC1B | DEP domain containing 1B | 3.0 | −0.33 |
| PTPRS | Protein tyrosine phosphatase, receptor type, S | 3.0 | 0.22 |
| HMMR | Hyaluronan-mediated motility receptor (RHAMM) | 3.0 | −0.39 |
| RPL38 | Ribosomal protein L38 | 3.0 | 0.16 |
| MCOLN2 | Mucolipin 2 | 3.0 | −0.17 |
| BUB1 | Budding uninhibited by benzimidazoles 1 homolog (yeast) | 3.0 | −0.31 |
| CLIC5 | Chloride intracellular channel 5 | 3.0 | −0.19 |
| C16orf5 | Official Symbol: CDIP1 and Name: cell death-inducing | 3.0 | 0.11 |
| | p53 target 1 | | |
| MAD1L1 | MAD1 mitotic arrest deficient-like 1 (yeast) | 2.9 | 0.14 |
| OLFM2 | Olfactomedin 2 | 2.9 | 0.15 |
| CLSPN | Claspin | 2.9 | −0.29 |
| FAM72B | Family with sequence similarity 72, member B | 2.9 | −0.28 |
| C1orf198 | Chromosome 1 open reading frame 198 | 2.9 | 0.16 |
| RPS15 | Ribosomal protein S15 | 2.9 | 0.15 |
| PHLDB3 | Pleckstrin homology-like domain, family B, member 3 | 2.9 | 0.14 |
| LOC96610 | BMS1 homolog, ribosome assembly protein (yeast) | 2.9 | −0.26 |
| | pseudogene | | |
| USP46 | Ubiquitin specific peptidase 46 | 2.9 | −0.15 |
| UHRF1 | Ubiquitin-like with PHD and ring finger domains 1 | 2.8 | −0.20 |
| ATAD2 | ATPase family, AAA domain containing 2 | 2.8 | −0.14 |
| DDX11L9 | DEAD/H (Asp-Glu-Ala-Asp/His) box helicase 11 like 9 | 2.8 | 0.51 |
| CDC25A | Cell division cycle 25 homolog A (S. pombe) | 2.8 | −0.39 |
| WWTR1 | WW domain containing transcription regulator 1 | 2.8 | −0.35 |
| NCAPH | Non-SMC condensin I complex, subunit H | 2.8 | −0.31 |
| CDCA2 | Cell division cycle associated 2 | 2.8 | −0.35 |
| PTPN13 | Protein tyrosine phosphatase, non-receptor type 13 (APO- | 2.8 | −0.23 |
| | 1/CD95 (Fas)-associated phosphatase) | | |
| DBP | D site of albumin promoter (albumin D-box) binding | 2.8 | 0.11 |
| | protein | | |
| CLDND1 | Claudin domain containing 1 | 2.8 | −0.12 |
| SLC39A4 | Solute carrier family 39 (zinc transporter), member 4 | 2.8 | 0.16 |
| APOA2 | Apolipoprotein A-II | 2.8 | −0.39 |
| SMAD1 | SMAD family member 1 | 2.8 | −0.21 |
| SMPD1 | Sphingomyelin phosphodiesterase 1, acid lysosomal | 2.7 | 0.11 |
| CMTM1 | CKLF-like MARVEL transmembrane domain containing 1 | 2.7 | −0.22 |
| MANEA | Mannosidase, endo-alpha | 2.7 | −0.17 |
| TSPAN33 | Tetraspanin 33 | 2.7 | 0.16 |
| C9orf16 | Chromosome 9 open reading frame 16 | 2.7 | 0.14 |
| CD7 | CD7 molecule | 2.7 | 0.13 |
| SLC9A3 | Solute carrier family 9, subfamily A (NHE3, cation proton | 2.7 | 0.30 |
| | antiporter 3), member 3 | | |
| FXYD2 | FXYD domain containing ion transport regulator 2 | 2.7 | 0.30 |
| KIF18A | Kinesin family member 18A | 2.7 | −0.23 |
| PDCD1LG2 | Programmed cell death 1 ligand 2 | 2.7 | −0.43 |
| IGF1 | Insulin-like growth factor 1 (somatomedin C) | 2.7 | −0.47 |
| CCDC101 | Coiled-coil domain containing 101 | 2.7 | 0.11 |
| LOC401242 | Uncharacterized LOC401242 | 2.7 | 0.17 |
| VEGFB | Vascular endothelial growth factor B | 2.7 | 0.12 |
| SLED1 | Proteoglycan 3 pseudogene | 2.7 | −0.39 |
| DHFR | Dihydrofolate reductase | 2.7 | −0.13 |
| ZWINT | ZW10 interactor | 2.7 | −0.25 |
| TOP2A | Topoisomerase (DNA) II alpha 170 kDa | 2.7 | −0.30 |
| NRP2 | Neuropilin 2 | 2.7 | 0.28 |
| TTK | TTK protein kinase | 2.7 | −0.31 |
| LOC402160 | Uncharacterized LOC402160 | 2.7 | −0.33 |
| EDAR | Ectodysplasin A receptor | 2.7 | 0.20 |
| TNXA | Tenascin XA (pseudogene) | 2.7 | 0.32 |
| SHISA3 | Shisa homolog 3 (Xenopus laevis) | 2.7 | −0.44 |
| FRG1B | FSHD region gene 1 family, member B | 2.6 | 0.18 |
| C16orf13 | Chromosome 16 open reading frame 13 | 2.6 | 0.12 |
| MCM4 | Minichromosome maintenance complex component 4 | 2.6 | −0.18 |
| PYCR2 | Pyrroline-5-carboxylate reductase family, member 2 | 2.6 | 0.08 |
| TSKU | Tsukushi, small leucine rich proteoglycan | 2.6 | 0.31 |
| GTSE1 | G-2 and S-phase expressed 1 | 2.6 | −0.29 |
| SLC22A17 | Solute carrier family 22, member 17 | 2.6 | 0.24 |
| C1orf116 | Chromosome 1 open reading frame 116 | 2.6 | 0.36 |
| PRRT1 | Proline-rich transmembrane protein 1 | 2.6 | 0.24 |
| PRTG | Protogenin | 2.6 | −0.27 |
| ZSCAN18 | Zinc finger and SCAN domain containing 18 | 2.6 | 0.13 |
| PLXDC1 | Plexin domain containing 1 | 2.6 | 0.17 |
| CLEC2L | C-type lectin domain family 2, member L | 2.6 | 0.45 |
| C9orf152 | Chromosome 9 open reading frame 152 | 2.6 | −0.37 |
| ALDOC | Aldolase C, fructose-bisphosphate | 2.6 | 0.12 |
| MIXL1 | Mix paired-like homeobox | 2.6 | −0.39 |
| NETO2 | Neuropilin (NRP) and tolloid (TLL)-like 2 | 2.6 | −0.15 |
| C9orf150 | Official Symbol: LURAP1L: and Name: leucine rich | 2.6 | 0.37 |
| | adaptor protein 1-like | | |
| FAM20A | Family with sequence similarity 20, member A | 2.6 | −0.32 |
| DHRS3 | Dehydrogenase/reductase (SDR family) member 3 | 2.6 | 0.14 |
| IGJ | Immunoglobulin J polypeptide, linker protein for | 2.6 | −0.38 |
| | immunoglobulin alpha and mu polypeptides | | |
| PERP | PERP, TP53 apoptosis effector | 2.6 | −0.24 |
| FBXO16 | F-box protein 16 | 2.6 | −0.38 |
| EIF3C | Eukaryotic translation initiation factor 3, subunit C | 2.6 | 0.88 |
| DMC1 | DMC1 dosage suppressor of mck1 homolog, meiosis- | 2.5 | −0.37 |
| | specific homologous recombination (yeast) | | |
| CCNA2 | Cyclin A2 | 2.5 | −0.23 |
| TNIP3 | TNFAIP3 interacting protein 3 | 2.5 | −0.28 |
| KIF2C | Kinesin family member 2C | 2.5 | −0.27 |
| C11orf2 | Official Symbol: VPS51 and Name: vacuolar protein | 2.5 | 0.10 |
| | sorting 51 homolog (S. cerevisiae) | | |
| LOC100128252 | Uncharacterized LOC100128252 | 2.5 | 0.23 |
| MPL | Myeloproliferative leukemia virus oncogene | 2.5 | 0.25 |
| NEK2 | NIMA-related kinase 2 | 2.5 | −0.35 |
| PHTF1 | Putative homeodomain transcription factor 1 | 2.5 | −0.14 |
| PARD3 | Par-3 partitioning defective 3 homolog (C. elegans) | 2.5 | 0.25 |
| LOC285954 | INHBA-AS1 INHBA antisense RNA 1 | 2.5 | 0.28 |
| KIF15 | Kinesin family member 15 | 2.5 | −0.27 |
| RPL36 | Ribosomal protein L36 | 2.5 | 0.15 |
| RPL23A | Ribosomal protein L23a | 2.5 | 0.14 |
| MTRNR2L1 | MT-RNR2-like 1 | 2.5 | 0.23 |
| ELL2 | Elongation factor, RNA polymerase II, 2 | 2.5 | −0.18 |
| MTRR | 5-methyltetrahydrofolate-homocysteine methyltransferase | 2.5 | −0.10 |
| | reductase | | |
| ANLN | Anillin, actin binding protein | 2.5 | −0.31 |
| RGS10 | Regulator of G-protein signaling 10 | 2.5 | 0.15 |
| CDCA5 | Cell division cycle associated 5 | 2.5 | −0.29 |
| CDCA7 | Cell division cycle associated 7 | 2.5 | −0.19 |
| PTCRA | Pre T-cell antigen receptor alpha | 2.5 | 0.30 |
| MTHFD2 | Methylenetetrahydrofolate dehydrogenase (NADP+ | 2.5 | −0.16 |
| | dependent) 2, methenyltetrahydrofolate cyclohydrolase | | |
| RRM2 | Ribonucleotide reductase M2 | 2.5 | −0.33 |
| ZFHX4 | Zinc finger homeobox 4 | 2.5 | −0.31 |
| ALDH1L2 | Aldehyde dehydrogenase 1 family, member L2 | 2.5 | −0.29 |
| UBE2J1 | Ubiquitin-conjugating enzyme E2, J1 | 2.5 | −0.14 |
| C1orf86 | Chromosome 1 open reading frame 86 | 2.4 | 0.11 |
| NLRP7 | NLR family, pyrin domain containing 7 | 2.4 | −0.24 |
| KRI1 | KRI1 homolog (S. cerevisiae) | 2.4 | 0.08 |
| ATXN7L2 | Ataxin 7-like 2 | 2.4 | 0.10 |
| CD3E | CD3e molecule, epsilon (CD3-TCR complex) | 2.4 | 0.12 |
| ESAM | Endothelial cell adhesion molecule | 2.4 | 0.25 |
| GRAP2 | GRB2-related adaptor protein 2 | 2.4 | 0.11 |
| RPL13 | Ribosomal protein L13 | 2.4 | 0.15 |
| RPL19 | Ribosomal protein L19 | 2.4 | 0.14 |
| NUSAP1 | Nucleolar and spindle associated protein 1 | 2.4 | −0.21 |
| PLK1 | Polo-like kinase 1 | 2.4 | −0.25 |
| LBH | Limb bud and heart development | 2.4 | 0.10 |
| NT5M | 5′,3′-nucleotidase, mitochondrial | 2.4 | 0.30 |
| TMEM8B | Transmembrane protein 8B | 2.4 | 0.11 |
| C6orf211 | Chromosome 6 open reading frame 211 | 2.4 | −0.12 |
| RAB25 | RAB25, member RAS oncogene family | 2.4 | 0.27 |
| TBK1 | TANK-binding kinase 1 | 2.4 | −0.13 |
| CCDC106 | Coiled-coil domain containing 106 | 2.4 | 0.13 |
| BRCA2 | Breast cancer 2, early onset | 2.4 | −0.19 |
| CHST14 | Carbohydrate (N-acetylgalactosamine 4-0) sulfotransferase | 2.4 | 0.09 |
| | 14 | | |
| RPL18A | Ribosomal protein L18a | 2.4 | 0.14 |
| SCUBE2 | Signal peptide, CUB domain, EGF-like 2 | 2.4 | −0.35 |
| CARD8 | Caspase recruitment domain family, member 8 | 2.4 | −0.10 |
| MIR3690 | microRNA 3690 | 2.4 | −0.36 |
| RPL28 | Ribosomal protein L28 | 2.4 | 0.13 |
| TLE2 | Transducin-like enhancer of split 2 (E(sp1) homolog, | 2.4 | 0.15 |
| | Drosophila) | | |
| RPL37A | Ribosomal protein L37a | 2.4 | 0.16 |
| KPNA7 | Karyopherin alpha 7 (importin alpha 8) | 2.4 | −0.27 |
| CADM1 | Cell adhesion molecule 1 | 2.4 | −0.27 |
| USE1 | Unconventional SNARE in the ER 1 homolog (S. cerevisiae) | 2.4 | 0.11 |
| SGK223 | Homolog of rat pragma of Rnd2 | 2.4 | 0.12 |
| CENPF | Centromere protein F, 350/400 kDa (mitosin) | 2.4 | −0.20 |
| CDC42EP1 | CDC42 effector protein (Rho GTPase binding) 1 | 2.4 | 0.30 |
| LRRC14B | Leucine rich repeat containing 14B | 2.4 | 0.31 |
| THAP7 | THAP domain containing 7 | 2.4 | 0.11 |
| KIF14 | Kinesin family member 14 | 2.4 | −0.32 |
| LTBP3 | Latent transforming growth factor beta binding protein 3 | 2.4 | 0.14 |
| C19orf33 | Chromosome 19 open reading frame 33 | 2.4 | 0.39 |
| DDX51 | DEAD (Asp-Glu-Ala-Asp) box polypeptide 51 | 2.4 | 0.09 |
| CLSTN3 | Calsyntenin 3 | 2.4 | −0.13 |
| COL6A2 | Collagen, type VI, alpha 2 | 2.4 | 0.19 |
| PTPN22 | Protein tyrosine phosphatase, non-receptor type 22 | 2.4 | −0.11 |
| | (lymphoid) | | |
| CENPE | Centromere protein E, 312 kDa | 2.3 | −0.25 |
| GNAZ | Guanine nucleotide binding protein (G protein), alpha z | 2.3 | 0.26 |
| | polypeptide | | |
| AK5 | Adenylate kinase 5 | 2.3 | 0.18 |
| POU5F1 | POU class 5 homeobox 1 | 2.3 | −0.22 |
| GPR146 | G protein-coupled receptor 146 | 2.3 | 0.23 |
| LAT | Linker for activation of T cells | 2.3 | 0.11 |
| NOS3 | Nitric oxide synthase 3 (endothelial cell) | 2.3 | 0.15 |
| MYLPF | Myosin light chain, phosphorylatable, fast skeletal muscle | 2.3 | 0.29 |
| BRCA1 | Breast cancer 1, early onset | 2.3 | −0.14 |
| NCRNA00200 | LINC00200 long intergenic non-protein coding RNA 200 | 2.3 | 0.49 |
| PILRB | Paired immunoglobin-like type 2 receptor beta | 2.3 | 0.10 |
| MIR650 | microRNA 650 | 2.3 | −0.29 |
| SALL2 | Sal-like 2 (Drosophila) | 2.3 | 0.15 |
| CHMP7 | Charged multivesicular body protein 7 | 2.3 | 0.10 |
| FAM172BP | Family with sequence similarity 172, member B, | 2.3 | −0.26 |
| | pseudogene | | |
| C14orf101 | Chromosome 14 open reading frame 101 | 2.3 | −0.10 |
| GALNT14 | UDP-N-acetyl-alpha-D-galactosamine:polypeptide N- | 2.3 | −0.38 |
| | acetylgalactosaminyltransferase 14 (GalNAc-T14) | | |
| C20orf203 | Chromosome 20 open reading frame 203 | 2.3 | 0.31 |
| MIR2277 | microRNA 2277 | 2.3 | −0.37 |
| ZNF414 | Zinc finger protein 414 | 2.3 | 0.10 |
| C14orf148 | Official Symbol: NOXRED1 and Name: NADP-dependent | 2.3 | −0.20 |
| | oxidoreductase domain containing 1 | | |
| FAH | Fumarylacetoacetate hydrolase (fumarylacetoacetase) | 2.3 | 0.14 |
| PNMA6D | Paraneoplastic Ma antigen family member 6D | 2.3 | 0.51 |
| MOCS1 | Molybdenum cofactor synthesis 1 | 2.3 | 0.24 |
| RPS12 | Ribosomal protein S12 | 2.3 | 0.16 |
| ANKRD10 | Ankyrin repeat domain 10 | 2.3 | −0.07 |
| DGCR11 | DiGeorge syndrome critical region gene 11 (non-protein | 2.3 | −0.16 |
| | coding) | | |
| TRIM28 | Tripartite motif containing 28 | 2.3 | 0.08 |
| SLC30A8 | Solute carrier family 30 (zinc transporter), member 8 | 2.3 | −0.30 |
| SERPINE2 | Serpin peptidase inhibitor, clade E (nexin, plasminogen | 2.3 | 0.22 |
| | activator inhibitor type 1), member 2 | | |
| PLK4 | Polo-like kinase 4 | 2.3 | −0.21 |
| FAM178B | Family with sequence similarity 178, member B | 2.3 | 0.28 |
| CD38 | CD38 molecule | 2.3 | −0.20 |
| SNORA24 | Small nucleolar RNA, H/ACA box 24 | 2.3 | −0.31 |
| MAF | V-maf musculoaponeurotic fibrosarcoma oncogene | 2.3 | −0.14 |
| | homolog (avian) | | |
| TYMS | Thymidylate synthetase | 2.3 | −0.28 |
| NDUFA3 | NADH dehydrogenase (ubiquinone) 1 alpha subcomplex, 3, | 2.3 | 0.13 |
| | 9 kDa | | |
| FLT3LG | Fms-related tyrosine kinase 3 ligand | 2.3 | 0.11 |
| CDC6 | Cell division cycle 6 homolog (S. cerevisiae) | 2.3 | −0.31 |
| NOG | Noggin | 2.3 | 0.18 |
| LRP2BP | LRP2 binding protein | 2.3 | −0.19 |
| BTN2A1 | Butyrophilin, subfamily 2, member A1 | 2.3 | −0.09 |
| SAMD14 | Sterile alpha motif domain containing 14 | 2.3 | 0.43 |
| WASF3 | WAS protein family, member 3 | 2.3 | 0.41 |
| NLGN2 | Neuroligin 2 | 2.3 | 0.17 |
| OST4 | Oligosaccharyltransferase 4 homolog (S. cerevisiae) | 2.3 | 0.14 |
| TFAP4 | Transcription factor AP-4 (activating enhancer binding | 2.3 | 0.09 |
| | protein 4) | | |
| VSIG2 | V-set and immunoglobulin domain containing 2 | 2.2 | 0.31 |
| EXO1 | Exonuclease 1 | 2.2 | −0.28 |
| ID3 | Inhibitor of DNA binding 3, dominant negative helix-loop- | 2.2 | 0.12 |
| | helix protein | | |
| TPX2 | TPX2, microtubule-associated, homolog (Xenopus laevis) | 2.2 | −0.27 |
| INTS1 | Integrator complex subunit 1 | 2.2 | 0.09 |
| CACNA1E | Calcium channel, voltage-dependent, R type, alpha 1E | 2.2 | −0.37 |
| | subunit | | |
| BANF1 | Barrier to autointegration factor 1 | 2.2 | 0.10 |
| RPS19 | Ribosomal protein S19 | 2.2 | 0.14 |
| REG4 | Regenerating islet-derived family, member 4 | 2.2 | 0.30 |
| GNA12 | Guanine nucleotide binding protein (G protein) alpha 12 | 2.2 | 0.11 |
| GSG2 | Germ cell associated 2 (haspin) | 2.2 | −0.24 |
| PLS3 | Plastin 3 | 2.2 | −0.25 |
| SEMA6C | Sema domain, transmembrane domain (TM), and | 2.2 | 0.14 |
| | cytoplasmic domain, (semaphorin) 6C | | |
| DUSP5 | Dual specificity phosphatase 5 | 2.2 | −0.17 |
| KNTC1 | Kinetochore associated 1 | 2.2 | −0.11 |
| FCGBP | Fc fragment of IgG binding protein | 2.2 | 0.24 |
| TXNDC5 | Thioredoxin domain containing 5 (endoplasmic reticulum) | 2.2 | −0.33 |
| IFT140 | Intraflagellar transport 140 homolog (Chlamydomonas) | 2.2 | 0.11 |
| GAMT | Guanidinoacetate N-methyltransferase | 2.2 | 0.14 |
| GATSL3 | GATS protein-like 3 | 2.2 | 0.10 |
| ZBTB46 | Zinc finger and BTB domain containing 46 | 2.2 | 0.12 |
| GLYATL1 | Glycine-N-acyltransferase-like 1 | 2.2 | 0.33 |
| KIAA0408 | KIAA0408 | 2.2 | −0.50 |
| TRPC2 | Transient receptor potential cation channel, subfamily C, | 2.2 | 0.32 |
| | member 2, pseudogene | | |
| OPN1SW | Opsin 1 (cone pigments), short-wave-sensitive | 2.2 | −0.23 |
| TMEM25 | Transmembrane protein 25 | 2.2 | 0.13 |
| TXNDC11 | Thioredoxin domain containing 11 | 2.2 | −0.11 |
| SL42 | Src-like-adaptor 2 | 2.2 | 0.10 |
| CDH24 | Cadherin 24, type 2 | 2.2 | 0.16 |
| IL12A | Interleukin 12A (natural killer cell stimulatory factor 1, | 2.2 | −0.21 |
| | cytotoxic lymphocyte maturation factor 1, p35) | | |
| ALKBH7 | AlkB, alkylation repair homolog 7 (E. coli) | 2.2 | 0.12 |
| TMEM177 | Transmembrane protein 177 | 2.2 | 0.13 |
| C14orf132 | Chromosome 14 open reading frame 132 | 2.2 | 0.43 |
| KCNAB1 | Potassium voltage-gated channel, shaker-related subfamily, | 2.2 | −0.17 |
| | beta member 1 | | |
| IL11RA | Interleukin 11 receptor, alpha | 2.2 | 0.12 |
| RPL29 | Ribosomal protein L29 | 2.2 | 0.13 |
| ZNF80 | Zinc finger protein 80 | 2.2 | −0.20 |
| ESCO2 | Establishment of cohesion 1 homolog 2 (S. cerevisiae) | 2.2 | −0.28 |
| CAPN13 | Calpain 13 | 2.2 | −0.39 |
| ZNF517 | Zinc finger protein 517 | 2.2 | 0.10 |
| CYP46A1 | Cytochrome P450, family 46, subfamily A, polypeptide 1 | 2.2 | 0.32 |
| HRASLS | HRAS-like suppressor | 2.2 | 0.35 |
| DTL | Denticleless E3 ubiquitin protein ligase homolog | 2.2 | −0.31 |
| | (Drosophila) | | |
| PLLP | Plasmolipin | 2.2 | 0.24 |
| EPHX1 | Epoxide hydrolase 1, microsomal (xenobiotic) | 2.2 | 0.09 |
| DPY19L3 | Dpy-19-like 3 (C. elegans) | 2.2 | −0.11 |
| MIR1914 | microRNA 1914 | 2.2 | 0.32 |
| C20orf11 | Official Symbol: GID8 and Name: GID complex subunit 8 | 2.2 | 0.07 |
| | homolog (S. cerevisiae) | | |
| DDX11L2 | DEAD/H (Asp-Glu-Ala-Asp/His) box helicase 11 like 2 | 2.2 | 0.38 |
| CETN2 | Centrin, EF-hand protein, 2 | 2.2 | 0.11 |
| NRGN | Neurogranin (protein kinase C substrate, RC3) | 2.2 | 0.30 |
| IRF2BP1 | Interferon regulatory factor 2 binding protein 1 | 2.1 | 0.09 |
| FHIT | Fragile histidine triad | 2.1 | 0.23 |
| WTIP | Wilms tumor 1 interacting protein | 2.1 | −0.26 |
| RASGRP2 | RAS guanyl releasing protein 2 (calcium and DAG- | 2.1 | 0.07 |
| | regulated) | | |
| SLCO4A1 | Solute carrier organic anion transporter family, member | 2.1 | −0.21 |
| | 4A1 | | |

### Illustrative Embodiments

In some implementations, the present disclosure is directed to methods, apparatus, medical profiles and kits useful for distinguishing between or among at least two conditions for diagnosis and/or risk assessment of an individual suspected of having or observed as having atypical development, wherein the at least two conditions comprise autism spectrum disorder (ASD) and developmental delay not due to autism spectrum disorder (DD).

To improve evaluation, in some implementations, a number of additional factors may be considered in combination with the evaluation of the expression profile. For example, an algorithm for obtaining a risk score, a likelihood, a diagnosis, or other such determination may involve one or more of: additional biochemical markers, patient parameters, patient demographic parameters, and/or patient biophysical measurements. Demographic parameters, in some examples, include age, ethnicity, current medications, and/or the like. Patient biophysical measurements, in some examples, include weight, body mass index (BMI), blood pressure, heart rate, cholesterol levels, triglyceride levels, medical conditions, and/or the like.

Turning to FIG. 1, a flow chart illustrates an example method 100 for distinguishing between or among at least two conditions for diagnosis and/or risk assessment of an individual suspected of having or observed as having atypical development, according to some embodiments. Steps of the method 100, may be performed, for example, using a software algorithm and using a diagnostic kit.

In some implementations, the method begins with 102 obtaining a blood sample from an individual suspected or observed (e.g., by a medical practitioner) as having atypical development (e.g., developmental delay of some kind) Step 104 is measurement of the expression level of a specific, predetermined set of genes of the blood sample from the individual. In certain embodiments, measurement is performed using next generation sequencing apparatus and software (e.g., using RNA-Seq). Step 106 is inputting measured expression levels of the predetermined genes in a predetermined gene expression signature, where the signature may have been obtained from control samples of known diagnosis. Step 108 is display or otherwise retrieval of a score, likelihood, or diagnosis output from the gene expression signature indicating a more or less likely indication of ASD versus DD (or DD versus ASD).

In some implementations, the output is presented upon the display of a user computing device. In some implementations, the risk assessment score is presented as a read-out on a display portion of a specialty computing device (e.g., a test kit analysis device). The risk assessment score may be presented as a numeric value, bar graph, pie graph, or other illustration expressing a relative risk of the individual having ASD.

In some embodiments, demographic values and/or biophysical values are accessed and accounted for in the determination of the output in step 108.

The present disclosure also provides commercial packages, or kits, for measurement of the expression level of the set of genes needed for input in the gene expression signature, e.g., where such measurement is performed by a next generation sequencer.

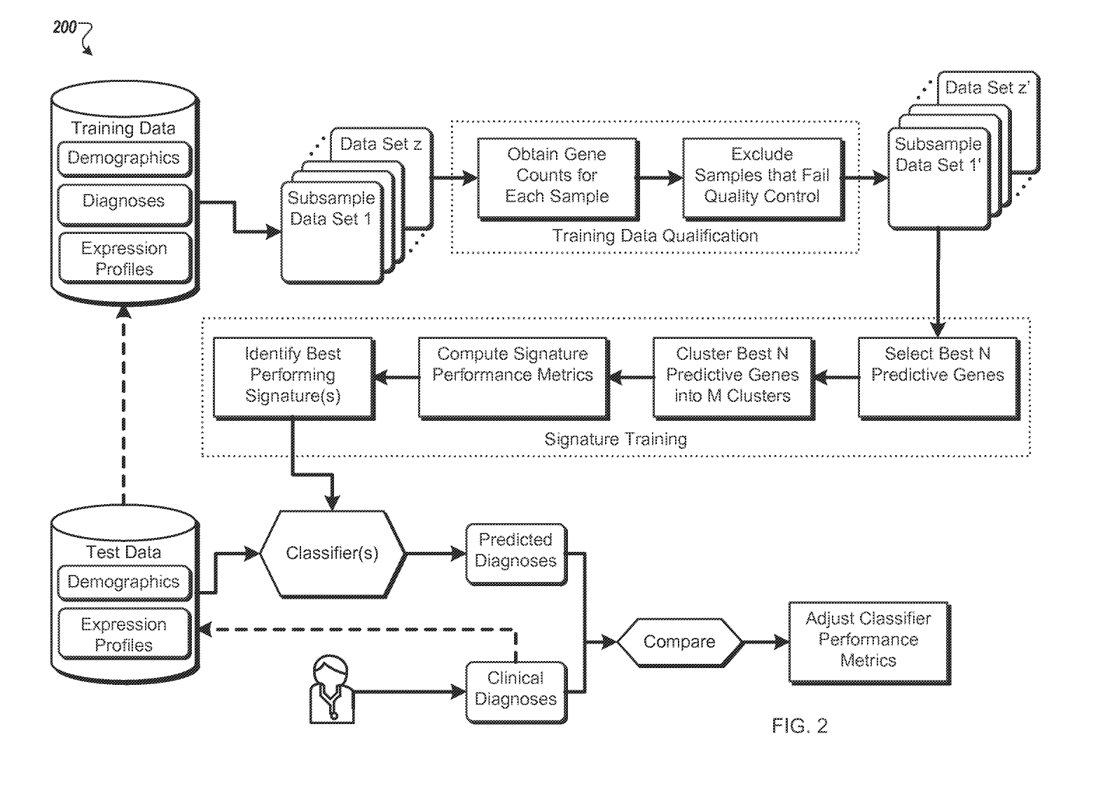

Turning to FIG. 2, an illustrative procedure is provided for determination of the classifier(s) described herein. Training data which includes gene expression profiles, known diagnoses, and, optionally, demographic information for each of a set of training samples, is used to determine the classifier(s). The training data is qualified by excluding samples that do not have a sufficiently high gene count. Signature training is performed on subsampled data sets. The best N predictive genes are selected and clustered into M clusters. Signature performance metrics are computed and the best performing signature(s) are identified and use to classify test data. The measured expression profile for a given sample is used as input in the classifier(s), and predicted diagnosis is determined therefrom. An additional step may include confirming diagnosis (e.g., by a medical practitioner) at the time of the predicted diagnosis, or later. For samples having known diagnosis, the predictive capability of the classifier(s) may be assessed, and the classifier adjusted.

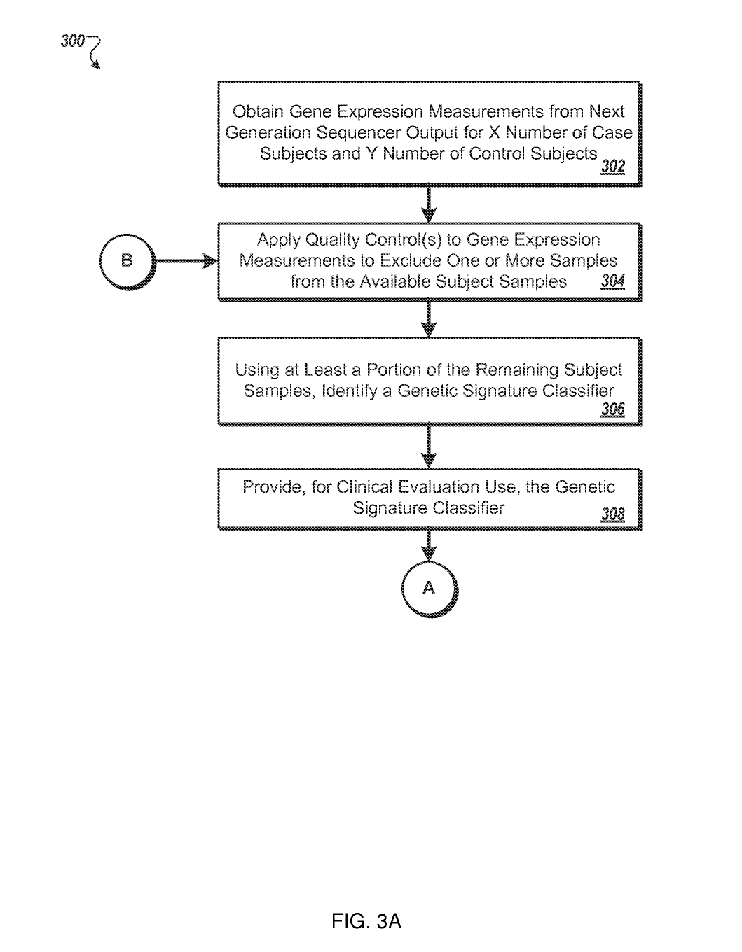

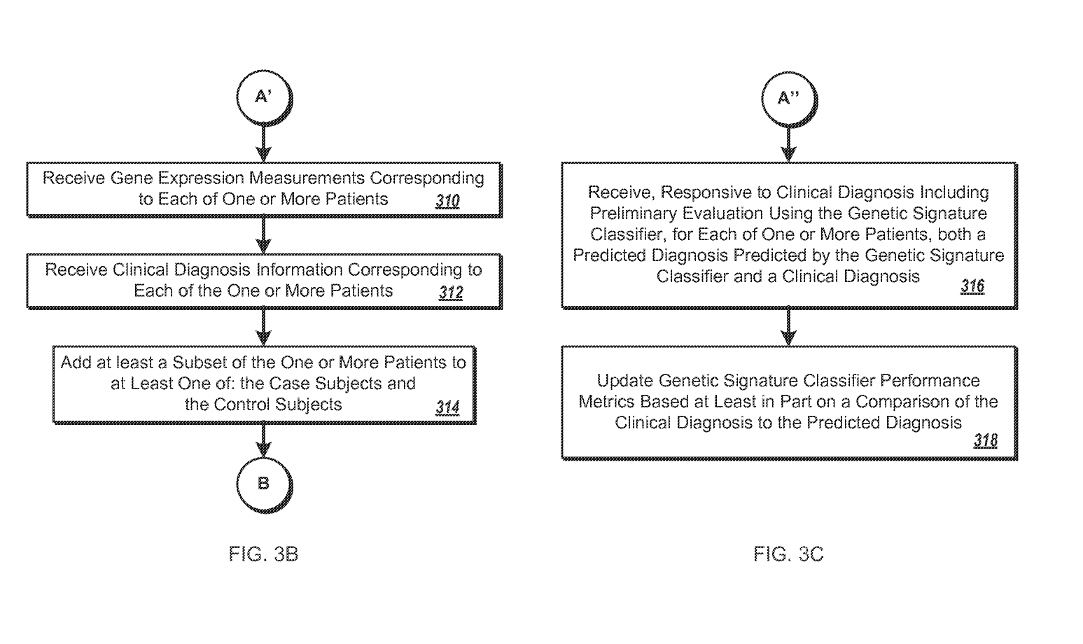

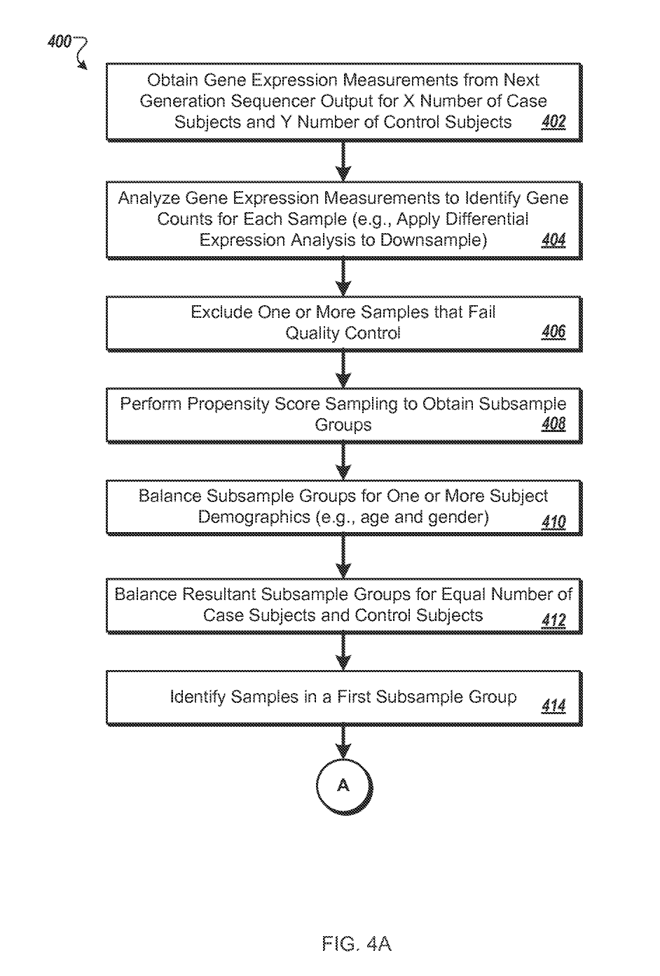

Turning to FIGS. 3A, 3B, and 3C, an example of a method of determining classifiers according to illustrative embodiments is described. In step 302, gene expression measurements are obtained from a next generation sequencer for X number of case subjects and Y number of control subjects. In step 304, quality control(s) is/are applied to gene expression measurements to exclude one or more samples from the available subject samples, e.g., if they have insufficient gene counts. In step 306, using at least a portion of the remaining (qualified) subject samples, a genetic signature classifier is determined/identified. Step 308 is providing the genetic signature classifier for clinical evaluation use.

In certain embodiments, feedback (B) from clinical use of the signature classifier may be used in the evolution of the signature(s) and/or development of new signatures. For example, predicted diagnoses may be confirmed or contradicted by a medical practitioner, and a comparison between predicted diagnoses and clinical diagnoses can be used as feedback in signature development. In FIG. 3B, gene expression measurements and corresponding clinical diagnoses for a set of patients are received (310, 312), and this set of patients may be considered case subjects and/or control subjects (314), e.g., in the signature training procedure of FIG. 2. In FIG. 3C, a clinical diagnosis and a diagnosis predicted by the current signature for a set of patients is received 316, and the genetic signature classifier performance metrics are updated using this data 318.

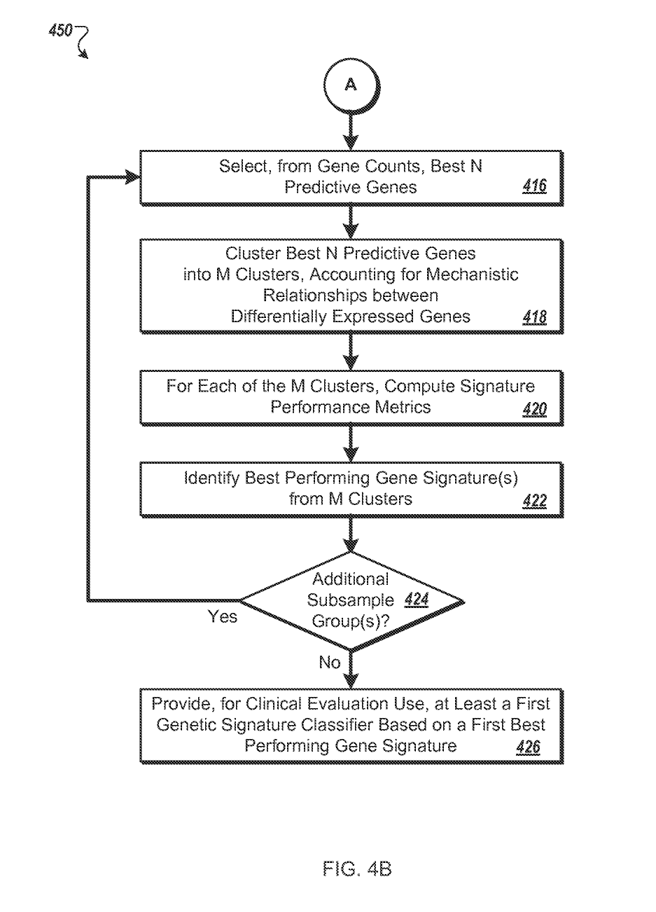

FIGS. 4A and 4B show an illustrative subsampling procedure 400 in the signature training method, according to some embodiments. Gene expression measurements are obtained from next generation sequencer output for X number of case subjects and Y number of control subjects 402. The gene expression measurements are analyzed 404 to identify gene counts for each sample, e.g., by applying differential expression analysis to downsample, rather than scale. Sample that fail this quality control (e.g., minimum gene count) are excluded (406). Step 408 is performance of propensity score sampling to determine subsample groups. Subsample groups are balanced (410) for one or more subject demographics (e.g., age and gender), and the resultant subsample groups may be balanced for equal number (or approximately equal number) of case subjects and control subjects, for example in step 412.

For each subsample group is identified in step 414, the best N predictive genes are selected in step 416. The best N predictive genes are clustered into M clusters in step 418, accounting for mechanistic relationships between differentially expressed genes. In step 420, for each of the M clusters, signature performance metrics are computed. The best performing gene signatures are identified from the M clusters in step 422. The process is repeated 424 for the next subsample group. Upon completion, one or more genetic signature classifiers are provided for clinical use, based on best performing gene signatures 426.

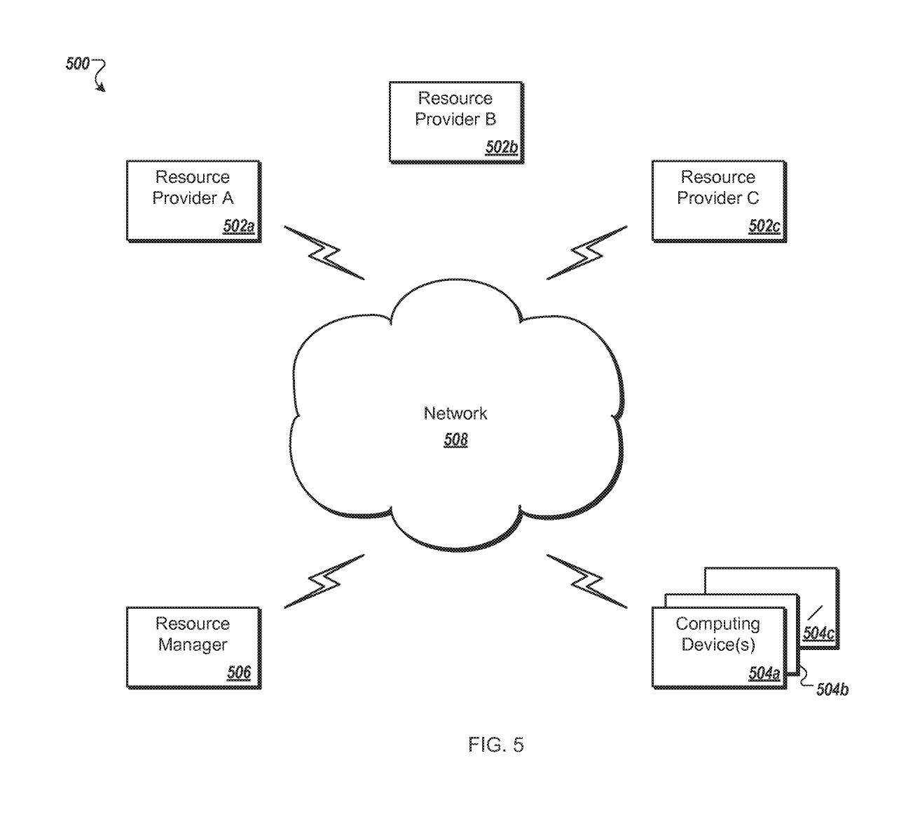

An implementation of an exemplary cloud computing environment 500 for use with the systems and methods described herein is shown in FIG. 5. The cloud computing environment 500 may include one or more resource providers 502a, 502b, 502c (collectively, 502). Each resource provider 502 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 502 may be connected to any other resource provider 502 in the cloud computing environment 500. In some implementations, the resource providers 502 may be connected over a computer network 508. Each resource provider 502 may be connected to one or more computing device 504a, 504b, 504c (collectively, 504), over the computer network 508.

The cloud computing environment 500 may include a resource manager 506. The resource manager 506 may be connected to the resource providers 502 and the computing devices 504 over the computer network 508. In some implementations, the resource manager 506 may facilitate the provision of computing resources by one or more resource providers 502 to one or more computing devices 504. The resource manager 506 may receive a request for a computing resource from a particular computing device 504. The resource manager 506 may identify one or more resource providers 502 capable of providing the computing resource requested by the computing device 504. The resource manager 506 may select a resource provider 502 to provide the computing resource. The resource manager 506 may facilitate a connection between the resource provider 502 and a particular computing device 504. In some implementations, the resource manager 506 may establish a connection between a particular resource provider 502 and a particular computing device 504. In some implementations, the resource manager 506 may redirect a particular computing device 504 to a particular resource provider 502 with the requested computing resource.

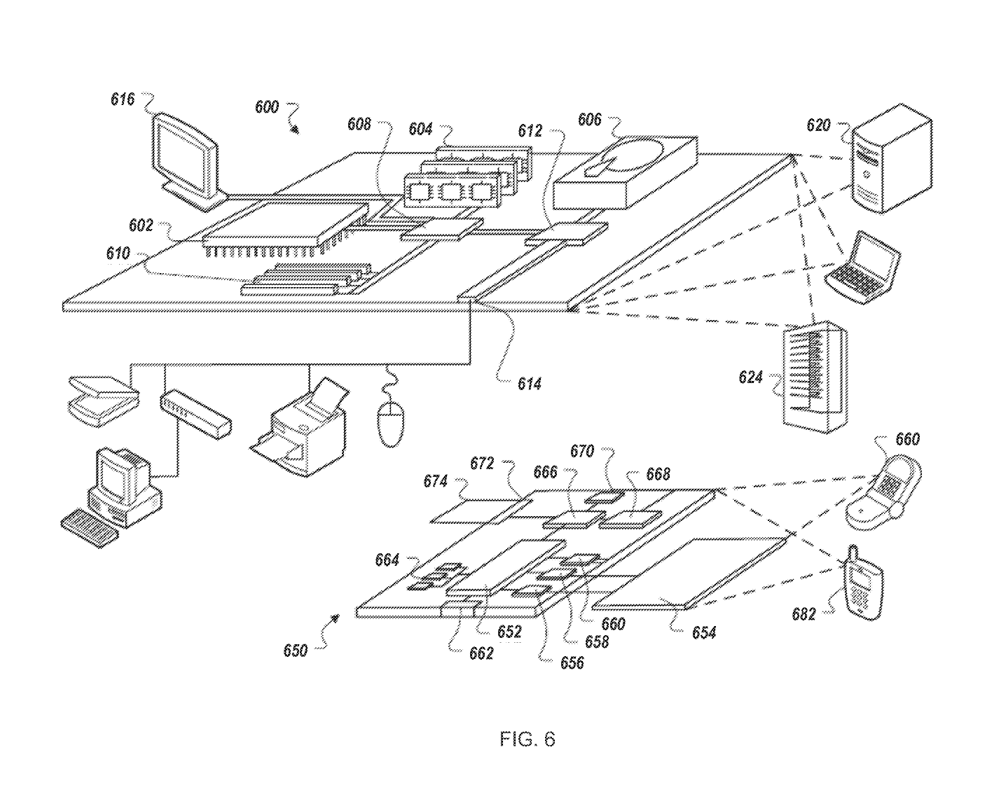

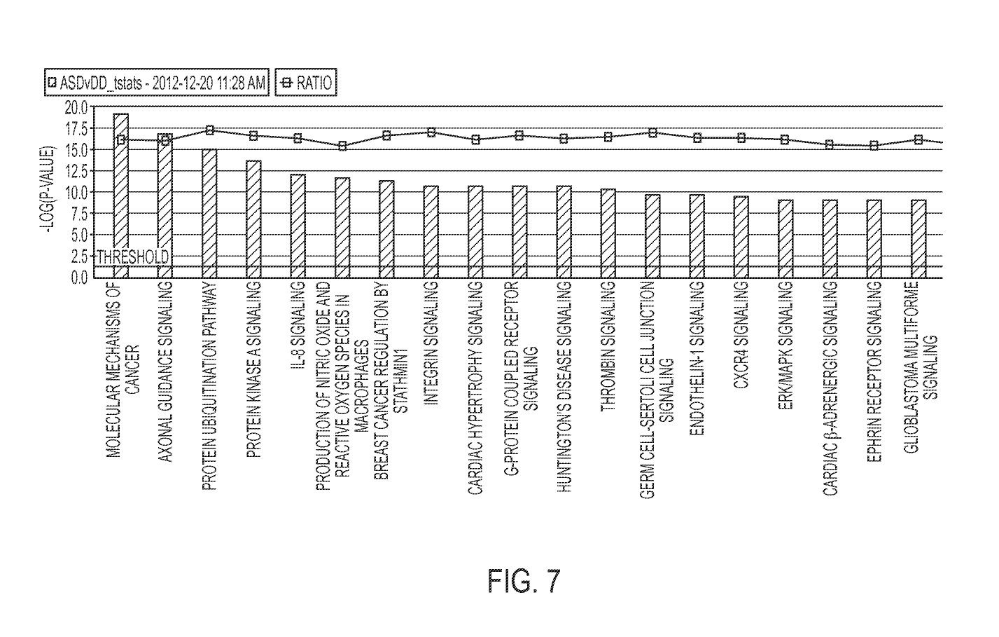

FIG. 6 shows an example of a computing device 600 and a mobile computing device 650 that can be used to implement the techniques described in this disclosure. The computing device 600 is intended to represent various forms of digital computers, such as laptops, desktops, workstations, personal digital assistants, servers, blade servers, mainframes, and other appropriate computers. The mobile computing device 650 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 600 includes a processor 602, a memory 604, a storage device 606, a high-speed interface 608 connecting to the memory 604 and multiple high-speed expansion ports 610, and a low-speed interface 612 connecting to a low-speed expansion port 614 and the storage device 606. Each of the processor 602, the memory 604, the storage device 606, the high-speed interface 608, the high-speed expansion ports 610, and the low-speed interface 612, are interconnected using various busses, and may be mounted on a common motherboard or in other manners as appropriate. The processor 602 can process instructions for execution within the computing device 600, including instructions stored in the memory 604 or on the storage device 606 to display graphical information for a GUI on an external input/output device, such as a display 616 coupled to the high-speed interface 608. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system).

The memory 604 stores information within the computing device 600. In some implementations, the memory 604 is a volatile memory unit or units. In some implementations, the memory 604 is a non-volatile memory unit or units. The memory 604 may also be another form of computer-readable medium, such as a magnetic or optical disk.

The storage device 606 is capable of providing mass storage for the computing device 600. In some implementations, the storage device 606 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 602), perform one or more methods, such as those described above. The instructions can also be stored by one or more storage devices such as computer- or machine-readable mediums (for example, the memory 604, the storage device 606, or memory on the processor 602).

The high-speed interface 608 manages bandwidth-intensive operations for the computing device 600, while the low-speed interface 612 manages lower bandwidth-intensive operations. Such allocation of functions is an example only. In some implementations, the high-speed interface 608 is coupled to the memory 604, the display 616 (e.g., through a graphics processor or accelerator), and to the high-speed expansion ports 610, which may accept various expansion cards (not shown). In the implementation, the low-speed interface 612 is coupled to the storage device 606 and the low-speed expansion port 614. The low-speed expansion port 614, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 600 may be implemented in a number of different forms, as shown in the figure. For example, it may be implemented as a standard server 620, or multiple times in a group of such servers. In addition, it may be implemented in a personal computer such as a laptop computer 622. It may also be implemented as part of a rack server system 624. Alternatively, components from the computing device 600 may be combined with other components in a mobile device (not shown), such as a mobile computing device 650. Each of such devices may contain one or more of the computing device 600 and the mobile computing device 650, and an entire system may be made up of multiple computing devices communicating with each other.

The mobile computing device 650 includes a processor 652, a memory 664, an input/output device such as a display 654, a communication interface 666, and a transceiver 668, among other components. The mobile computing device 650 may also be provided with a storage device, such as a micro-drive or other device, to provide additional storage. Each of the processor 652, the memory 664, the display 654, the communication interface 666, and the transceiver 668, are interconnected using various buses, and several of the components may be mounted on a common motherboard or in other manners as appropriate.

The processor 652 can execute instructions within the mobile computing device 650, including instructions stored in the memory 664. The processor 652 may be implemented as a chipset of chips that include separate and multiple analog and digital processors. The processor 652 may provide, for example, for coordination of the other components of the mobile computing device 650, such as control of user interfaces, applications run by the mobile computing device 650, and wireless communication by the mobile computing device 650.

The processor 652 may communicate with a user through a control interface 658 and a display interface 656 coupled to the display 654. The display 654 may be, for example, a TFT (Thin-Film-Transistor Liquid Crystal Display) display or an OLED (Organic Light Emitting Diode) display, or other appropriate display technology. The display interface 656 may include appropriate circuitry for driving the display 654 to present graphical and other information to a user. The control interface 658 may receive commands from a user and convert them for submission to the processor 652. In addition, an external interface 662 may provide communication with the processor 652, so as to enable near area communication of the mobile computing device 650 with other devices. The external interface 662 may provide, for example, for wired communication in some implementations, or for wireless communication in other implementations, and multiple interfaces may also be used.

The memory 664 stores information within the mobile computing device 650. The memory 664 can be implemented as one or more of a computer-readable medium or media, a volatile memory unit or units, or a non-volatile memory unit or units. An expansion memory 674 may also be provided and connected to the mobile computing device 650 through an expansion interface 672, which may include, for example, a SIMM (Single In Line Memory Module) card interface. The expansion memory 674 may provide extra storage space for the mobile computing device 650, or may also store applications or other information for the mobile computing device 650. Specifically, the expansion memory 674 may include instructions to carry out or supplement the processes described above, and may include secure information also. Thus, for example, the expansion memory 674 may be provide as a security module for the mobile computing device 650, and may be programmed with instructions that permit secure use of the mobile computing device 650. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier. that the instructions, when executed by one or more processing devices (for example, processor 652), perform one or more methods, such as those described above. The instructions can also be stored by one or more storage devices, such as one or more computer- or machine-readable mediums (for example, the memory 664, the expansion memory 674, or memory on the processor 652). In some implementations, the instructions can be received in a propagated signal, for example, over the transceiver 668 or the external interface 662.

The mobile computing device 650 may communicate wirelessly through the communication interface 666, which may include digital signal processing circuitry where necessary. The communication interface 666 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 668 using a radio-frequency. In addition, short-range communication may occur, such as using a Bluetooth®, Wi-Fi™, or other such transceiver (not shown). In addition, a GPS (Global Positioning System) receiver module 670 may provide additional navigation- and location-related wireless data to the mobile computing device 650, which may be used as appropriate by applications running on the mobile computing device 650.

The mobile computing device 650 may also communicate audibly using an audio codec 660, which may receive spoken information from a user and convert it to usable digital information. The audio codec 660 may likewise generate audible sound for a user, such as through a speaker, e.g., in a handset of the mobile computing device 650. Such sound may include sound from voice telephone calls, may include recorded sound (e.g., voice messages, music files, etc.) and may also include sound generated by applications operating on the mobile computing device 650.

The mobile computing device 650 may be implemented in a number of different forms, as shown in the figure. For example, it may be implemented as a cellular telephone 580. It may also be implemented as part of a smart-phone 682, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In view of the structure, functions and apparatus of the systems and methods described here, in some implementations, a systems, methods, and apparatus for distinguishing between or among at least two conditions (e.g., ASD and DD) for diagnosis and/or risk assessment of an individual suspected of having or observed as having atypical development are provided. Having described certain implementations of methods, systems, and apparatus herein, it will now become apparent to one of skill in the art that other implementations incorporating the concepts of the disclosure may be used. Therefore, the disclosure should not be limited to certain implementations, but rather should be limited only by the spirit and scope of the following claims.

1-5. (canceled)

6. A method for distinguishing between two conditions for diagnosis of an individual suspected of having or observed as having atypical development, wherein the two conditions comprise autism spectrum disorder (ASD) and developmental delay not due to autism spectrum disorder (DD), the method comprising steps of:
obtaining a sample of peripheral blood from the individual;
measuring levels of analytes in the sample corresponding to the metabolism of at least one of tetrahydrofolate, prostaglandin, prostanoid, ribonucleoside diphosphate, cholesterol hydrogen peroxide, and glycerol-3-phosphate;
determining a likelihood that the individual has ASD or DD based on the levels of analytes measured in the sample.

7. The method of claim 6 wherein the individual is 5 years old or less.

8. The method of claim 6 wherein the individual is 3 years old or less.

9. The method of claim 6 wherein the analyte is mRNA.

10. The method of claim 9 wherein the mRNA is derived from at least one of C20orf173, TRPM5, TPM2, CCNE2, CKAP2L, CAND2, MTRNR2L3, LDLRAP1, ASPM, ZDHHC15, RASL10B, ST8SIA1, CLEC12B, MARCKSL1, SHCBP1, DEPDC1, TSHR, NCAPG, RPLP2, CENPA, SORBS3, MCM10, HELLS, RNF208, E2F8, PTK7, GRM3, CPSF1, and CDHR1.
